(19) United States
(12) Patent Application Publication
(10) Pub. No.: US 2026/0179729 A1
(43) Pub. Date: Jun. 25, 2026

(54) METHOD OF TRAINING POTENTIAL ENERGY MODEL AND ELECTRONIC DEVICE FOR PERFORMING THE SAME

(71) Applicant: SAMSUNG ELECTRONICS CO., LTD., SUWON-SI (KR)

(72) Inventors: Jiali PANG, Xi'an (CN); Lin CHEN, Xi'an (CN); Zhen ZHANG, Xi'an (CN); Ihor VASYLTSOV, Suwon-si Gyeonggi-do (KR); Zehao CHEN, Xi'an (CN)

(21) Appl. No.: 19/389,381

(22) Filed: Nov. 14, 2025

(30) Foreign Application Priority Data
Dec. 19, 2024 (CN) 202411877529.1
Apr. 21, 2025 (KR) 10-2025-0051954

(51) Int. Cl. G16C 10/00

(52) U.S. Cl. CPC G16C 10/00

(57) ABSTRACT

A system, method, and electronic device for training and simulating a potential energy model includes obtaining a key sample from a plurality of samples of a data set for a molecular dynamics (MD) simulation, and, based on the key sample, determining a first cutoff radius of the potential energy model. The system then, using a first atomic graph generated based on the key sample and the first cutoff radius, executes training of the potential energy model, and determines a second cutoff radius different from the first cutoff radius based on an inference result of the potential energy model trained using the first atomic graph.

### CROSS-REFERENCE TO RELATED APPLICATIONS

This application claims priority under 35 USC § 119 (a) to Chinese Patent Application No. 202411877529.1, filed on Dec. 19, 2024, in the China National Intellectual Property Administration, and further to Korean Patent Application No. 10-2025-0051954, filed on Apr. 21, 2025, in the Korean Intellectual Property Office, the disclosures of which are incorporated by reference herein in their entirety.

### TECHNICAL FIELD

The present disclosure relates to artificial intelligence, and more particularly, to a method of training a potential energy model by determining a hyperparameter of a graph neural network (GNN)-based potential energy model for a molecular dynamics (MD) simulation, an electronic device configured to perform the method, and a non-transitory computer-readable storage medium storing code that, when executed by at least one processor, causes the processor to perform one or more functions related to the method.

### DISCUSSION OF THE RELATED ART

An MD simulation is a technique that may be used to study the properties and behavior of materials by simulating the motion of atoms and/or molecular particles. Recently, advances in artificial intelligence have enabled different implementations of MD simulation techniques.

The present embodiments include a GNN-based potential energy model which may be used for an MD simulation. The potential energy model may receive input information such as the coordinates of atoms, the types of atoms, and the distance between atoms, and output the energy and force of each atom. The GNN-based potential energy model may generate a graph that represents a state of the molecular system by forming edges between atoms based on the distance between atoms.

The above information is presented as a technical background to help with the understanding of the disclosure. The above should not be construed as an admission that any of the described information constitutes prior art with respect to the present disclosure.

### SUMMARY

According to some aspects, the present embodiments include an electronic device with at least one processor including processing circuitry and a memory including one or more storage media storing instructions. When executed by the at least one processor individually or collectively, the instructions cause the electronic device to perform obtaining a key sample from a plurality of samples of a data set for a molecular dynamics (MD) simulation. The instructions may further cause the electronic device to determine, based on the key sample, a first cutoff radius of a potential energy model. The device may then execute training of the potential energy model using a first atomic graph generated based on the key sample and the first cutoff radius. Additionally, the instructions may cause the electronic device to determine a second cutoff radius different from the first cutoff radius based on an inference result of the trained potential energy model. The inference result includes an energy parameter and a force parameter of an atom.

According to some aspects, the obtaining of the key sample may include obtaining the key sample from the plurality of samples of the data set using a weighted support vector regressor (SVR).

Each of the plurality of samples may include one or more atoms. In some implementations, obtaining the key sample using the weighted SVR includes determining a weight corresponding to each of the plurality of samples based on statistical characteristics of the plurality of samples, obtaining a plurality of weighted samples by applying the weight to each of the plurality of samples, and obtaining, as the key sample, a support vector from the plurality of weighted samples using the weighted SVR.

According to some aspects, the determining of the weight corresponding to each sample may include determining a count of one or more samples having a same or similar number of atoms among the plurality of samples of the data set, computing a ratio of this count to the total number of samples as the statistical characteristics, and determining the weight corresponding to each sample based on this ratio. The ratio may be computed by dividing the count of the one or more samples having the same or similar number of atoms by a total number of the plurality of samples.

In some implementations, the determining of the first cutoff radius of the potential energy model includes determining, for each elemental combination of atoms of the key sample, an importance value corresponding to a degree to which each elemental combination contributes to a number of edges of an atomic graph, and determining the first cutoff radius of each elemental combination of the atoms based on the determined importance value of each elemental combination of the atoms.

In some implementations, the determining of the importance value includes determining an environmental descriptor of each of the atoms of the key sample, computing an element ratio of a neighboring atom within a reference cutoff radius of each of the atoms of the key sample, and determining, based on the environmental descriptor and the element ratio of the neighboring atom of each of the atoms, the importance value of each elemental combination of the atoms.

In some implementations, the determining of the environmental descriptor of each of the atoms of the key sample includes generating, based on position coordinates and force coordinates of the respective atom and position coordinates and force coordinates of neighboring atoms within the reference cutoff radius of the respective atom, the environmental descriptor of each of the atoms.

In some implementations, the computing of the element ratio of the neighboring atom within the reference cutoff radius of each of the atoms of the key sample includes determining a number of neighboring atoms corresponding to each element within the reference cutoff radius of each of the atoms of the key sample, determining a total number of the neighboring atoms within the reference cutoff radius of each of the atoms, and computing, as the element ratio of the neighboring atom corresponding to the each element, a ratio of the number of neighboring atoms corresponding to the each element to the total number of the neighboring atoms within the reference cutoff radius of each of the atoms.

In some implementations, the determining of the importance value of each elemental combination of the atoms, based on the environmental descriptor and the element ratio of the neighboring atom of each of the atoms, includes: determining, based on environmental descriptors of atoms of a first sample of a plurality of samples of the key sample, a defined number of atoms among the atoms of the first sample, and determining, based on an element ratio of a neighboring atom of a second element type to each of one or more atoms of a first element type among the defined number of atoms of the first sample, the importance value of an elemental combination corresponding to the first element type and the second element type. The second element type may be same as or different from the first element type.

In some implementations, the determining of the cutoff radius of each elemental combination of the atoms includes determining, based on the importance value of each elemental combination of the atoms, the cutoff radius of each elemental combination within a defined cutoff radius range. In some implementations, determining the second cutoff radius based on the inference result of the trained potential energy model includes based on the inference result, the first cutoff radius, and the key sample, determining a loss and based on the loss, determining the second cutoff radius.

The instructions, when executed by the at least one processor individually or collectively, may cause the electronic device to further perform: generating, based on the second cutoff radius, a second atomic graph as an input to the trained potential energy model, finetuning, using the second atomic graph, the trained potential energy model, and obtaining a final potential energy model by repeatedly updating the second cutoff radius, generating a second atomic graph based on the updated second cutoff radius, and finetuning the trained potential energy model until an inference result of the potential energy model satisfies a defined requirement for an MD simulation.

According to some aspects, embodiments further include a method performed by an electronic device, the method including obtaining a key sample from a plurality of samples of a data set for an MD simulation. The method may include, based on the key sample, determining a first cutoff radius of a potential energy model. The method may include, executing, using a first atomic graph generated based on the key sample and the first cutoff radius, training of the potential energy model. The method may include determining a second cutoff radius different from the first cutoff radius based on an inference result of the potential energy model trained using the first atomic graph. The inference result of the potential energy model may include an energy parameter and a force parameter of an atom.

According to another aspect, embodiments include a non-transitory computer-readable storage medium storing one or more programs including instructions that, when executed by at least one processor of an electronic device individually or collectively, may cause the electronic device to perform obtaining a key sample from a plurality of samples of a data set for an MD simulation. The instructions, when executed by at least one processor of an electronic device individually or collectively, may cause the electronic device to perform, based on the key sample, determining a first cutoff radius of a potential energy model. The instructions, when executed by at least one processor of an electronic device individually or collectively, may cause the electronic device to perform executing training of the potential energy model using a first atomic graph generated based on the key sample and the first cutoff radius. The instructions, when executed by at least one processor of an electronic device individually or collectively, may cause the electronic device to perform determining a second cutoff radius different from the first cutoff radius based on an inference result of the potential energy model trained using the first atomic graph. The inference result of the potential energy model may include an energy parameter and a force parameter of an atom.

Additional aspects of the present embodiments will be set forth in following the description, or will be apparent from the description, or may be learned by practice of the disclosure.

### BRIEF DESCRIPTION OF THE DRAWINGS

These and other aspects, features, and advantages of the invention will become apparent and more readily appreciated from the following description of embodiments, taken in conjunction with the accompanying drawings, in which:

FIG. 1 illustrates a flowchart for determining a hyperparameter of a graph neural network (GNN)-based potential energy model, according to an embodiment;

FIG. 2 illustrates an example of a method of determining a hyperparameter of a GNN-based potential energy model, according to an embodiment;

FIG. 3 illustrates a flowchart for determining a hyperparameter of a GNN-based potential energy model, according to an embodiment;

FIGS. 4A, 4B, and 4C are diagrams which illustrate an atomic graph according to some embodiments;

FIG. 5 illustrates an example of a method of determining a hyperparameter of a GNN-based potential energy model, according to an embodiment;

FIG. 6A illustrates an example of a process for obtaining a key sample, according to an embodiment;

FIG. 6B illustrates a flowchart for obtaining a key sample according to an embodiment;

FIG. 7 illustrates an example of a process of determining weights of samples, according to an embodiment;

FIG. 8 illustrates an example of a method of obtaining a key sample using a weighted support vector regressor (SVR), according to an embodiment;

FIG. 9 illustrates changes in a neighborhood of SiN molecules before and after a cutoff radius is adjusted, according to an embodiment;

FIG. 10 illustrates a flowchart for determining a cutoff radius of each elemental combination, according to an embodiment;

FIG. 11 illustrates a block diagram of an electronic device according to an embodiment;

FIG. 12 illustrates a block diagram of an electronic device configured to determine a hyperparameter of a GNN-based potential energy model according to an embodiment; and

FIG. 13 illustrates a flowchart for iteratively updating a cutoff radius according to an embodiment.

### DETAILED DESCRIPTION

The following structural or functional descriptions of embodiments are provided as examples only, and various alterations and modifications may be made. Accordingly, the embodiments should not be construed as limited to the specific disclosure but should be understood to include all changes, equivalents, and replacements within the scope and spirit of the disclosure.

Although terms, such as first, second, and the like, may be used herein to describe various components, these terms should be used only to distinguish one component from another component. For example, a first component may be referred to as a second component, and similarly the second component may also be referred to as the first component.

It should be noted that if one component is described as being “connected,” “coupled,” or “joined” to another component, a third component may be “connected,” “coupled,” and “joined” between the first and second components, although the first component may be directly connected, coupled, or joined to the second component.

The singular forms “a,” “an,” and “the” are intended to include the plural forms as well, unless the context clearly indicates otherwise. It will be further understood that the terms “comprises/comprising” and/or “includes/including” when used herein, specify the presence of stated features, integers, steps, operations, elements, components, or groups thereof, but do not preclude the presence or addition of one or more other features, integers, steps, operations, elements, components, or groups thereof.

Unless otherwise defined, all terms used herein including technical or scientific terms have the same meaning as commonly understood by one of ordinary skill in the art to which this disclosure pertains. Terms, such as those defined in commonly used dictionaries, should be construed to have meanings matching with contextual meanings in the relevant art, and are not to be construed to have an ideal or excessively formal meaning unless otherwise defined herein.

Hereinafter, embodiments are described in detail with reference to the accompanying drawings. When describing the embodiments with reference to the accompanying drawings, the same or similar components may be denoted by the same reference numerals, and to the extent that descriptions of components are omitted, it will be appreciated that description of the same or similar components may be found elsewhere throughout the specification.

There are various techniques for a molecular dynamics (MD) simulation. For example, one method for implanting an MD simulation includes a neural equivariant interatomic potentials method. These methods use machine learning models to predict atomic forces and energies based on local atomic environments. They are able to learn learning complex many-body interactions from quantum mechanical calculations. Neural equivariant methods also product outputs that are invariant to rotations, translations, or reflections of the atomic system. An example of such a method is the NequIP method, which is an E (3)-equivariant graph neural network (GNN) method that learns interatomic potentials from a non-in-situ calculation of an MD simulation and may be based on a message passing mechanism. In some cases, however, neural equivariant interatomic potentials methods are unable to adaptively adjust training parameters (e.g., it is restricted to a single fixed cutoff radius value), and the presence of redundant information slows training, results in poor inference performance, and the method lacks computational parallelization capability. The present embodiments address these deficiencies by providing adaptive cutoff radius determination and improved training efficiency through key sample selection.

Other methods include strict local equivariant deep learning interatomic potentials method. These techniques maintain strict locality by ensuring that atomic interactions are computed based only on information within a defined neighborhood, which preserves physical principles while enabling efficient parallel computation. Additionally, each atom's environment can be processed independently without requiring global information exchange. The Allegro model is one example of such a method. These techniques focus on scalability through parallelization. However, some strict local equivariant deep learning methods may use excessive computational resources, or exhibit slow simulation processes and suboptimal inference performance that limit their practical applicability. The present embodiments, in contrast, implementing weighted sampling techniques and adaptive parameter optimization to reduce computational burden while maintaining high accuracy.

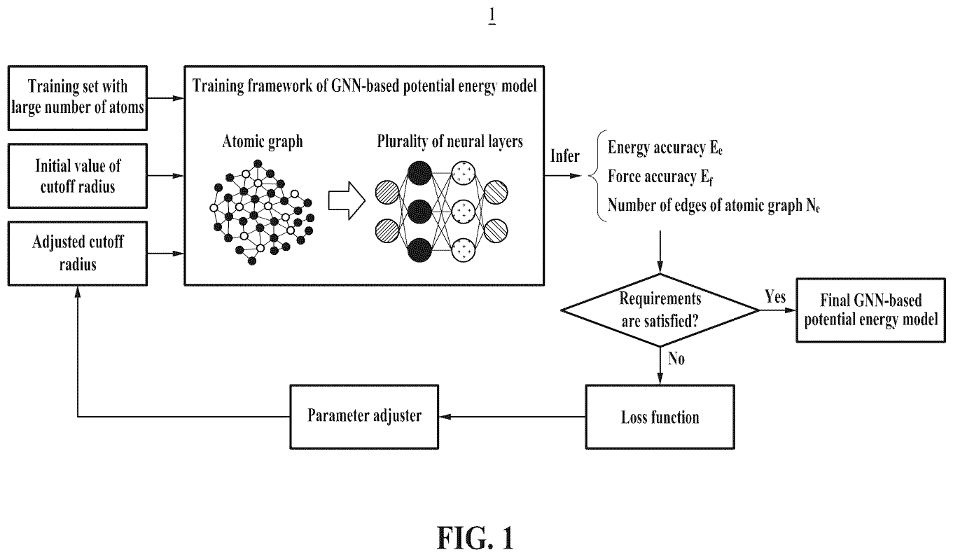

FIG. 1 illustrates a flowchart for determining a hyperparameter of a graph neural network (GNN)-based potential energy model, according to an embodiment.

Referring to FIG. 1, a system 1 may determine an atomic graph of the potential energy model by providing an initial cutoff radius 105, which is one hyperparameter of the model, and obtaining and providing a data set 100 (or training set) to the GNN-based potential energy model.

In general, a graph neural network (GNN) is a type of artificial neural network configured to process data that is structured as a graph including nodes and edges. In such a configuration, nodes may represent entities such as atoms, and edges may represent relationships such as interatomic distances or bonds. During training, the GNN performs message passing between connected nodes so that a feature vector of each node is updated based on features of neighboring nodes.

Through multiple iterations of message passing, the GNN encodes local and non-local interactions to enable accurate prediction of system-level properties. In the present embodiments, the atomic graph generated from the data set is input to the GNN so that the potential energy model can learn to infer energy and force parameters associated with the atoms.

The data set 100 (e.g., a training set) for model training may include cells including a large number of atoms. For example, the data set may include a molecular data set or an atomic data set. The cells may include unit cells. A unit cell represents the smallest repeating structural unit of a crystalline material that, when repeated in three dimensions, forms the complete crystal structure. For example, the data set may include a molecular data set containing various molecular configurations with their corresponding quantum mechanically calculated energies and forces, or an atomic data set containing different atomic arrangements and their associated properties. These data sets serve as ground truth references that enable the potential energy model to learn accurate interatomic relationships for subsequent MD simulations.

The system 1 may implement a training framework of the GNN-based potential energy model for training the potential energy model based on the determined atomic graph. The GNN-based potential energy model may include a plurality of neural layers. In a first phase, an atomic graph may be initialized from the data set 100 using the initial cutoff radius 105. After each training iteration, the atomic graph may be updated to the version obtained from the previous training iteration using the adjusted cutoff radius 110. The GNN-based potential energy model may include a plurality of neural layers that process the atomic graph structure. The repeated training iterations may be referred to herein as a “finetuning” process.

The potential energy model 115 may include both the atomic graph representation and a plurality of neural network layers that process the graph structure. The atomic graph defines the connectivity and relationships between atoms based on the cutoff radius, and the neural layers learn to extract features and predict system properties based on the state encoded in the atomic graph. An output from the potential energy model may include an energy parameter and a force parameter of an atom.

In an example according to FIG. 1, the initial value of the cutoff radius may include an arbitrary value, a preset value, or a value determined from preliminary analysis of the training data set 100.

In addition, a hyperparameter such as the cutoff radius of the GNN-based potential energy model may affect the scale and accuracy of the model. Therefore, embodiments implement an adaptively adjustable cutoff radius to optimize model performance. Furthermore, this allows for increased simulation speed of the GNN-based potential energy model.

After one or more training iterations of the potential energy model 115 within the training framework, the system 1 may determine inference results 120 such as energy accuracy Ee, force accuracy Ef, and edge number Ne of the atomic graph using the trained potential energy model. At decision block 125, the system 1 may determine whether the currently trained potential energy model satisfies the requirements for performing an MD simulation based on an inference result (e.g., inference parameters such as energy accuracy Ee, force accuracy Ef, and edge number Ne of the atomic graph) of the trained potential energy model. The criteria may include threshold values for energy prediction accuracy (e.g., mean absolute error below a specified tolerance), force prediction accuracy (e.g., root mean square error within acceptable limits), computational efficiency metrics (e.g., edge number Ne within an optimal range for balancing accuracy and speed), or convergence stability indicators.

The system 1 may determine the currently trained potential energy model as a final GNN-based potential energy model 115 as a final GNN-based potential energy model when the currently trained potential energy model 115 satisfies the defined criteria for performing an MD simulation.

At loss function block 135, the system 1 may calculate the loss of the currently trained potential energy model 115 based on the inference parameters when the currently trained potential energy model 115 does not satisfy the requirements for performing an MD simulation. At adjust parameters block 140, the system 1 may adjust a cutoff radius based on the calculated loss to generate the adjusted cutoff radius 110. The initialized cutoff radius may be referred to as a “first cutoff radius” herein, and the adjusted cutoff radius 110 may sometimes be referred to as a “second cutoff radius” herein. The system 1 may re-execute the training of the potential energy model based on the adjusted cutoff radius 110 to generate a GNN-based potential energy model that satisfies defined criteria for performing an MD simulation.

For example, the system 1 may calculate the loss of the potential energy model according to [Equation 1] below.

$$Loss = α {E}_{e} + β {E}_{f} + γ {N}_{e} \qquad ( 1 )$$

In [Equation 1], Loss may represent the loss of the trained potential energy model, α, β, and γ may represent the proportion (or weight) of corresponding inference parameter items (e.g., Ee, Er, or Ne) in the requirements for performing an MD simulation, respectively, Ee may represent energy accuracy, Er may represent force accuracy, and Ne may represent the number of edges of an atomic graph.

The system 1 may adjust a hyperparameter (e.g., cutoff radius) of the potential energy model using a parameter adjuster based on the calculation result of the loss. The parameter adjuster may employ gradient-based optimization techniques such as backpropagation to compute gradients of the loss function with respect to the cutoff radius and other model parameters. The parameter adjuster may utilize optimization algorithms such as Adam (Adaptive Moment Estimation), stochastic gradient descent (SGD), or RMSprop to update the neural network weights and biases within the plurality of neural layers of the potential energy model 115. Additionally, the parameter adjuster may implement learning rate scheduling, momentum updates, or adaptive learning rate mechanisms to enhance convergence stability and training efficiency. The adjusted cutoff radius 110 may be determined through this optimization process or may be a value that is selectively set based on the optimization results.

The system 1 may re-execute the training of the potential energy model 115 in the training framework using the adjusted cutoff radius and the data set 100 (e.g., a training set) for model training. After the re-training of the potential energy model is completed, the criteria discussed above for performing an MD simulation may or may not be met. If the criteria are not met, the system may proceed to perform another training iteration using the inference results 120 and the loss equation (e.g., Equation 1).

Training GNN-based potential energy models using data sets including a large number of atoms may involve substantial computational resources, and repetitive training processes due to fixed or arbitrary hyperparameter values (such as cutoff radius) may increase computational and time costs. The present embodiments address these challenges by implementing adaptive hyperparameter optimization and intelligent sampling techniques that reduce the computational burden while maintaining high accuracy. To improve convergence efficiency of loss calculations, the present embodiments consider both computational complexity and accuracy metrics, thereby achieving faster convergence compared to conventional methods that rely on fixed hyperparameters and full data set training.

The present embodiments include a method and device for determining a hyperparameter of the GNN-based potential energy model. The method and device for determining a hyperparameter of the GNN-based potential energy model may obtain a sample by sampling a data set for training a model, thereby reducing the amount of computation without compromising accuracy. The method and device for determining a hyperparameter of the GNN-based potential energy model may further reduce waste of unnecessary computational resources by setting an optimized hyperparameter using the sample obtained by sampling the data set.

The method and device for determining a hyperparameter of the GNN-based potential energy model adaptively adjust (e.g., initialize and then iteratively adjust) an optimal hyperparameter (e.g., cutoff radius) for a data set for training the model and may reduce the time cost required for adjusting the hyperparameter. The method and device for determining a hyperparameter of the GNN-based potential energy model may further train a model and adjust a hyperparameter by considering not only an accuracy index but also a computational amount index of the model through a balanced loss function.

Accordingly, the method and device for determining a hyperparameter of the GNN-based potential energy model according to an embodiment may achieve high accuracy during training of the model using an optimal hyperparameter (e.g., cutoff radius), shorten the time required for training, and reduce the computational amount.

The method and device for determining a hyperparameter of the GNN-based potential energy model may additionally accurately measure the influence of different elements on the number of edges of an atomic graph generated from the model in order to provide a guide to a user when a hyperparameter (e.g., cutoff radius) is adjusted.

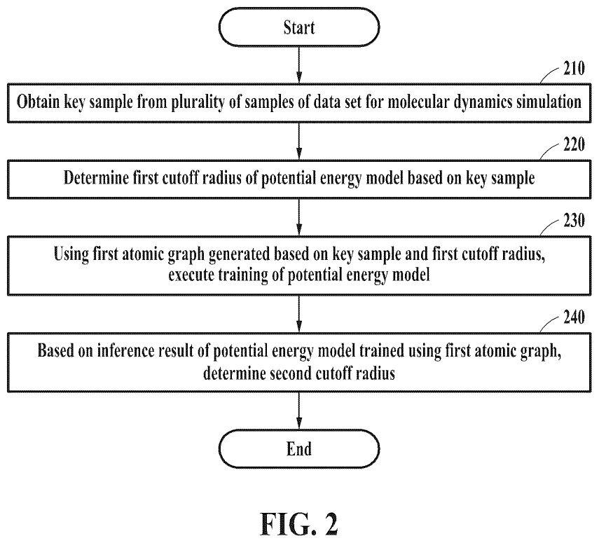

FIG. 2 illustrates an example of a method of determining a hyperparameter of a GNN-based potential energy model, according to an embodiment. FIG. illustrates a flowchart for determining a hyperparameter of a GNN-based potential energy model, according to an embodiment.

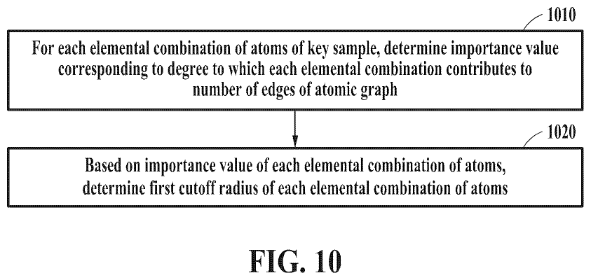

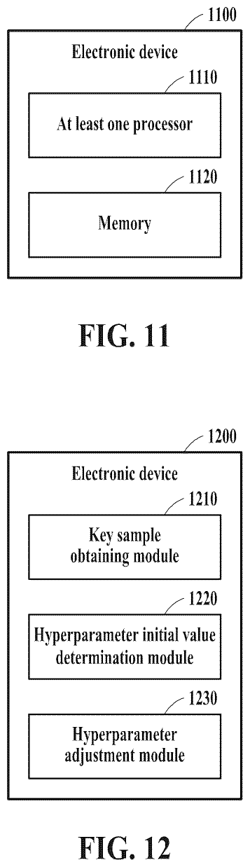

According to an embodiment, operations 210 to 240 may be performed by an electronic device (e.g., an electronic device 1100 of FIG. 11 or an electronic device 1200 of FIG. 12). The electronic device may include at least one processor including processing circuitry. The electronic device may include at least one memory including one or more storage media including instructions. The instructions, when executed by the at least one processor individually or collectively, may cause the electronic device to perform at least some of the operations related to the training method of the present disclosure. Accordingly, the electronic device constitutes a particular machine configured through the training process to store specific model parameters, cutoff radius values, and atomic graph configurations in the non-transitory memory. The stored instructions define a specific sequence of computational operations for adaptive hyperparameter optimization and weighted sampling that transforms the electronic device into a specialized system for molecular dynamics simulation modeling.

In the following embodiments, operations may be performed sequentially but not necessarily. For example, the order of the operations may change, and at least two of the operations may be performed in parallel.

In operation 210, the electronic device may obtain a key sample among a plurality of samples of a data set for an MD simulation. Rather than processing an entire data set as described in FIG. 1, some embodiments may determine a representative subset that maintains the structural distribution and statistical characteristics of the original data set while significantly reducing computational requirements. The electronic device may obtain a key sample having a data characteristic corresponding to the data set by sampling the data set (or an original data set). The key sample may include a smaller number of samples than the data set.

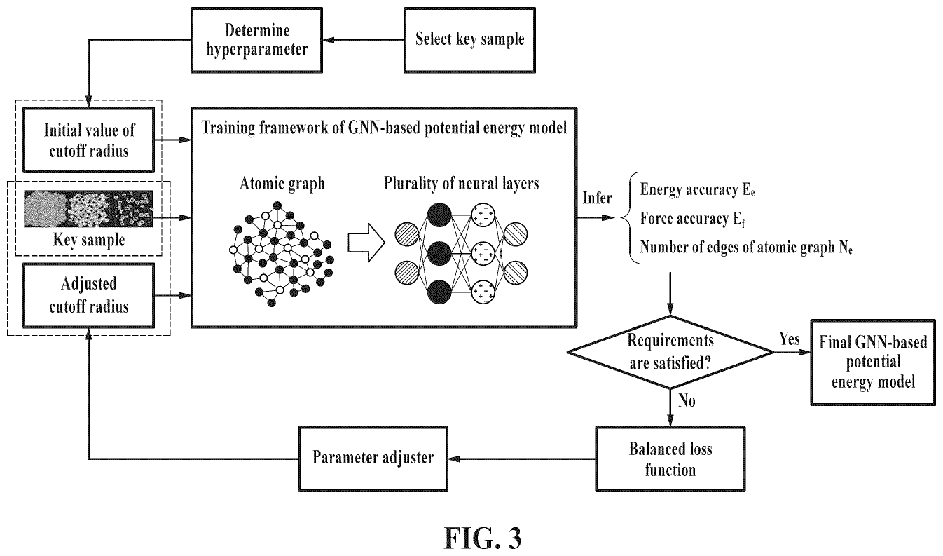

For example, referring to FIG. 3, the electronic device may determine a key sample (e.g., a key sample of FIG. 3) to be used for determining a hyperparameter and as an input to a GNN-based training framework among the plurality of samples of the data set by selecting the key sample. The key sample may be obtained using a weighted support vector regressor (SVR) that identifies support vectors defining the regression function, as will be described in detail with respect to FIGS. 3, 5, and 6A. The key sample may include a relatively small amount of data.

As described with reference to FIG. 1, a data set for an MD simulation may include a large number of atoms. The data set may include a molecular data set and/or an atomic data set. The data set may include a plurality of unit cells. Each unit cell may include one or more atoms. The number and distribution of atoms within each unit cell may be the same or different.

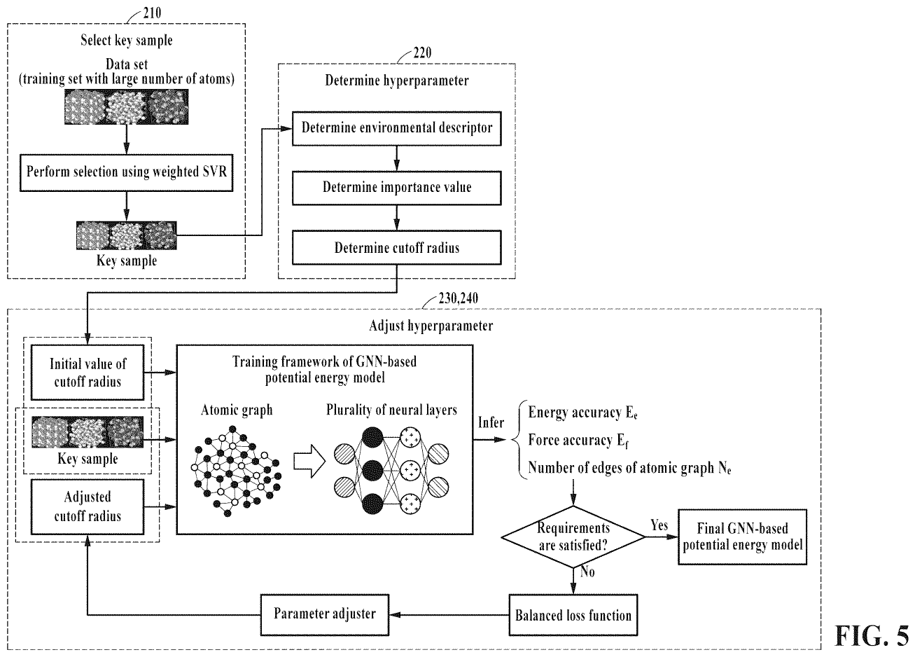

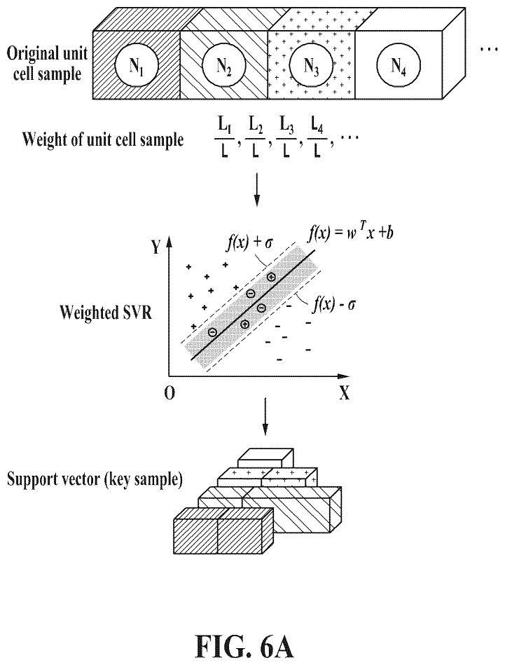

Each of the plurality of unit cells included in the data set may be one sample. For example, the data set including the plurality of unit cells may include a plurality of samples. In some cases, one sample may include a plurality of unit cells. The configuration of a sample is not limited to the example of the present disclosure, and a sample may be referred to as a “unit cell sample.” A method of obtaining a key sample from a plurality of samples of a data set for an MD simulation by selecting a key sample according to an embodiment is described in detail with reference to FIGS. 5, 6A, and 6B.

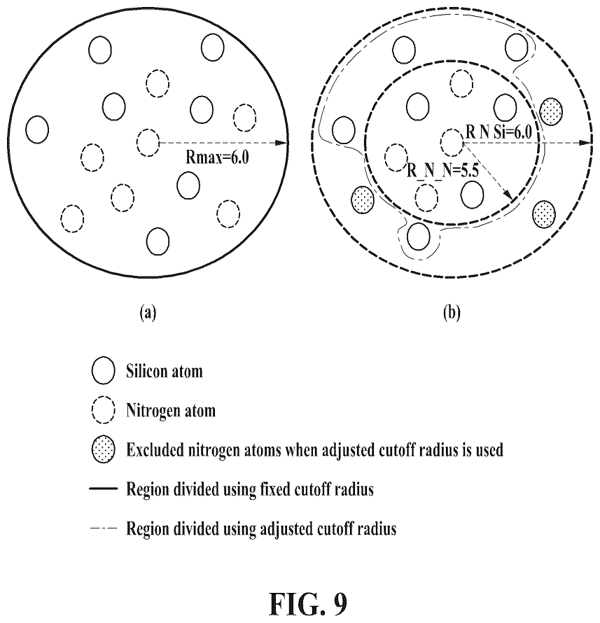

In operation 220, the electronic device may determine a cutoff radius (or an initial value of the cutoff radius) of the potential energy model based on the key sample. The cutoff radius defines the maximum distance at which atoms are considered to interact with each other in the molecular dynamics simulation, directly affecting both computational efficiency and model accuracy. By optimizing different cutoff radii for different elemental combinations, the system can capture meaningful atomic interactions while avoiding unnecessary computational overhead. The cutoff radius may include a cutoff radius of each elemental combination of the atoms of the key sample. The cutoff radius may represent a cutoff radius (or a value of the cutoff radius) for each of one or more different elemental combinations. For example, referring to FIG. 3, a hyperparameter including an optimized cutoff radius (or an initial value of the cutoff radius) may be determined using the key sample. A method of adaptively determining a cutoff radius for each of different elemental combinations by considering the characteristics of a data set (e.g., an atomic data set and/or a molecular data set) according to an embodiment is described in detail with reference to FIG. 10.

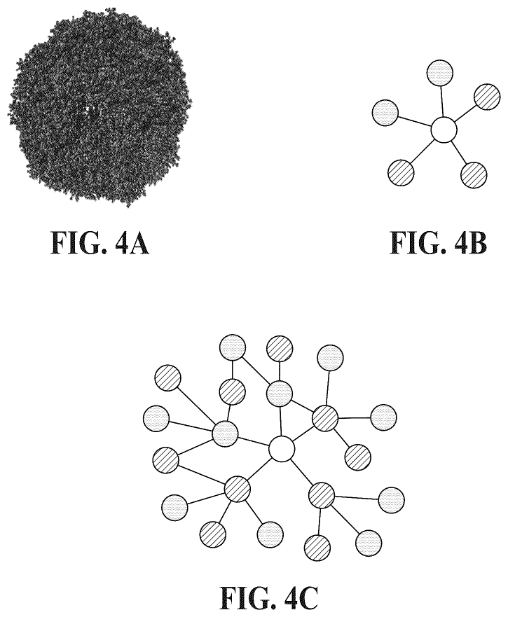

In operation 230, the electronic device may train the potential energy model using a first atomic graph generated based on the key sample and the cutoff radius. The atomic graph is described with reference to FIGS. 4A-4C.

FIG. 3 illustrates a flowchart for determining a hyperparameter of a GNN-based potential energy model, according to an embodiment. FIG. 3 illustrates a training framework similar to that shown in FIG. 1, however, the training set used in FIG. 3 may be significantly smaller than the training set used in FIG. 1. FIG. 3 will be discussed in detail after the following discussion of atomic graphs.

FIGS. 4A, 4B, and 4C are diagrams which illustrate an atomic graph according to some embodiments.

An electronic device may generate an atomic graph as an input of a potential energy model based on a key sample and a cutoff radius (or an initial value of the cutoff radius) in a training framework of a GNN-based potential energy model. In some embodiments, the atomic graph may be provided as an input to the potential energy model, while in other embodiments, the atomic graph may be incorporated as a part of the potential energy model structure. For example, in some embodiments, the atomic graph may be initialized from an atomic or molecular dataset and subsequently processed by the neural network layers of the potential energy model.

FIG. 4A illustrates a data set, e.g. an original data set. One small circle included in the data set may represent one atom. The data set may include data on an extremely large number of atoms or molecules, e.g. ranging from thousands to millions of atomic entities, which would be computationally prohibitive to process in their entirety using conventional training methods. The data set may include numerical data including atomic coordinates (position parameters), force vectors, energy values, elemental identities, and other physical properties associated with each atom or molecule, rather than visual representations. Each data point in the set may correspond to a specific atomic configuration within molecular structures or crystalline unit cells. Accordingly, the circular representations in FIG. 4A are provided as a schematic visualization of the vast quantity of atomic data contained within the original data set.

FIG. 4B may illustrate an atomic graph of a cutoff radius of one basic unit. The atomic graph may be used as an input of the potential energy model to predict the motion of atoms. The atomic graph may include nodes and edges.

Referring to FIG. 4B, one node (e.g., any circle in FIG. 4B) of the atomic graph may represent one atom of the data set. The atomic graph may include one edge (e.g., the line in FIG. 4B) between two atoms when the distance between the two atoms is smaller than the cutoff radius. The different fills of each circle may indicate different atoms.

In FIG. 4B, the value of the cutoff radius may be set to an arbitrary unit length. FIG. 4C may illustrate an atomic graph of a cutoff radius of, for example, two unit lengths. As the cutoff radius increases, the number of edges in the atomic graph may increase. Accordingly, with reference to FIG. 4C, the cutoff radius may have a significant effect on the number of edges in the atomic graph.

The atomic graph may include a significantly increased number of edges as the cutoff radius increases. Accordingly, the amount of data input to the potential energy model may increase. The number of edges in the atomic graph may directly correlate with computational complexity, as each edge represents an interatomic interaction that must be processed by the neural network layers. The larger the number of edges in the atomic graph, the greater the computational burden during both training and inference operations.

A method and device for determining a hyperparameter of a GNN-based potential energy model according to an embodiment may adaptively determine an optimal cutoff radius for a data set for training the model.

In operation 240, the electronic device may determine an adjusted cutoff radius based on an inference result of the potential energy model trained using a first atomic graph. A method of adjusting a hyperparameter is described in detail with reference to FIG. 5.

Referring again to FIG. 3, the electronic device may first select a key sample at block 300 and determine the cutoff radius hyperparameter at block 305 before providing the initial cutoff radius input to the GNN-based potential energy model 315. The electronic device may train the GNN-based potential energy model using a training framework of the GNN-based potential energy model based on an atomic graph (e.g., the first atomic graph) determined (e.g., initialized) from a key sample obtained from a training set, and the initial value of the cutoff radius.

The GNN-based potential energy model may include a plurality of neural layers. Additional detail regarding the updating the parameters of the plurality of neural layers is provided with reference to FIG. 1.

When the training of the GNN-based potential energy model is completed (e.g., at block 330 of the flowchart), the electronic device may determine inference parameters such as energy accuracy Ee, force accuracy Ef, and the number of edges Ne of the atomic graph using the currently trained potential energy model. The electronic device may determine whether the currently trained potential energy model satisfies the requirements for performing an MD simulation based on an inference result (e.g., inference parameters such as energy accuracy Ee, force accuracy Er, and the number of edges Ne of the atomic graph).

For example, criteria for performing an MD simulation may include thresholds and computational efficiency requirements that are determined or predetermined based on available computational resources. The computational efficiency requirements may account for hardware constraints such as memory capacity limitations, processing power restrictions, and available resources of specialized computing units including graphics processing units (GPUs) or tensor processing units (TPUs). These criteria ensure that the trained potential energy model can execute MD simulations within the operational parameters of the target computing environment.

The electronic device may determine that the currently trained potential energy model is final version of GNN-based potential energy model when the currently trained potential energy model satisfies the requirements for performing an MD simulation.

When the currently trained potential energy model satisfies the defined criteria for performing an MD simulation, the model may be deemed suitable for deployment in molecular dynamics simulations and can proceed to the model complete stage (e.g., block 330).

When the currently trained potential energy model does not satisfy the defined criteria for performing an MD simulation, the model is considered unsuitable for deployment due to insufficient accuracy, excessive computational demands, or incompatibility with available computing resources. In such cases, adjustment of hyperparameters (e.g., cutoff radius) of the potential energy model may be performed to obtain a potential energy model that meets the defined criteria for performing an MD simulation.

According to an embodiment, the electronic device may adjust the cutoff radius when determining, based on an inference result of the trained potential energy model using the first atomic graph, that the trained potential energy model does not satisfy the requirements for performing an MD simulation.

According to an embodiment, the electronic device may repeatedly perform the training operation of the potential energy model and re-determine the adjusted cutoff radius. The electronic device may generate a second atomic graph as an input to or part of the trained potential energy model using the first atomic graph based on the key sample and the adjusted cutoff radius. The electronic device may perform the training of the potential energy model using the second atomic graph.

For example, when the potential energy model trained using the second atomic graph satisfies the requirements for performing an MD simulation, the electronic device may determine the potential energy model trained using the second atomic graph as the final GNN-based potential energy model. If the potential energy model trained using the second atomic graph does not satisfy the requirements for performing an MD simulation, the electronic device may again readjust the cutoff radius. The electronic device may obtain the final potential energy model by repeating the determination of the adjusted cutoff radius, the generation of the second atomic graph (i.e., another atomic graph), and the training until the inference result of the potential energy model satisfies defined requirements for an MD simulation.

The requirements for performing an MD simulation may include, for example, user defined requirements for the potential energy model additionally or alternatively to the requirements described above. Embodiments are not limited thereto, and the requirements for performing an MD simulation may include any requirements that may be used to determine whether the final GNN-based potential energy model is obtained.

FIG. 5 illustrates an example of a method of determining a hyperparameter of a GNN-based potential energy model. FIG. 5 differs from the flowchart in FIG. 3 by providing detailed sub-operations within the main process blocks. Specifically, operation 210 from FIG. 2 is expanded to show detailed elements including a data set (training set with large number of atoms), a perform selection using weighted SVR operation, and the resulting key sample output.

The key sample is then input to block 220, which corresponds to operation 220 from FIG. 2 and includes a group of sub-operations: determine environmental descriptor operation, determine importance value operation, and determine cutoff radius operation. The output from the determine cutoff radius operation is subsequently input to the training framework, which is described above with reference to FIGS. 1 and 3 and corresponds to the operations 230 and 240 of FIG. 2.

According to an embodiment, operation 210 of obtaining the key sample among the plurality of samples of the data set for an MD simulation of FIG. 2 may include obtaining the key sample among the plurality of samples of the data set using a weighted support vector regressor (SVR). The electronic device may obtain the key sample by sampling the data set (or an original data set).

Referring to FIG. 5, the electronic device may determine the key sample by selecting some of the plurality of samples of the data set (e.g., a training set of a large number of atoms) using the weighted SVR. A method of obtaining a key sample using the weighted SVR is described in detail with reference to FIGS. 6A to 8. The electronic device may then determine and adjust a hyperparameter (such as the cutoff radius) based off the selected key sample (or N key samples) as described above with reference to FIGS. 1 and 3.

According to an embodiment, operation 220 of determining the cutoff radius of the potential energy model based on the key sample of FIG. 2 may include determining, for each elemental combination of the atoms of the key sample, the importance value corresponding to the degree to which each elemental combination contributes to the number of edges of the atomic graph and an operation of determining a cutoff radius of each elemental combination of the atoms based on the importance value of each elemental combination of the atoms. A method of determining a cutoff radius of each elemental combination is described in detail with reference to FIG. 10.

The electronic device may perform training of the potential energy model based on the key sample and the cutoff radius of each elemental combination. As described in operation 230 of FIG. 2, the electronic device may perform training of the potential energy model using an atomic graph (e.g., a first atomic graph) generated based on the key sample and the cutoff radius. As described in operation 240 of FIG. 2, the electronic device may adjust the cutoff radius based on an inference result of the trained potential energy model.

According to an embodiment, determining the adjusted cutoff radius based on the inference result of the trained potential energy model may include determining a loss based on the inference result, the cutoff radius (e.g., an initial value of the cutoff radius or a current cutoff radius), and the key sample, and further determining an adjusted cutoff radius based on the loss. For example, the electronic device may adjust a hyperparameter (e.g., a cutoff radius) using neural network intelligence (NNI).

According to an embodiment, the electronic device may determine a loss (Loss_{Balance}) through a loss function defined in [Equation 2].

$${Loss}_{Balance} = \frac{1}{N} {{\sum }_{i = 0}}^{N} ( α {‖ {E}_{i} - {Y}_{i}^{e} ‖}_{2}^{2} + β {‖ {F}_{i} - {Y}_{i}^{f} ‖}_{2}^{2} + γ \frac{\hat{C}}{{C}_{T}} + {{\sum }_{i = 0}}^{m} \frac{{w}_{i}}{{M}_{i}} \qquad ( 2 )$$

Each variable in [Equation 2] is described in [Table 1].

TABLE 1

| Variable | Description | Variable | Description |
|---|---|---|---|
| i | i-th atom | N | The total number of atoms |
| | | | included in a current unit cell |
| | | | sample |
| Eᵢ | The predicted energy of the i-th | γ | The ratio of calculation amount in |
| | atom | | requirements |
| Fᵢ | The predicted force of the i-th | Ĉ | The number of edges of an atomic |
| | atom | | graph determined based on a |
| | | | current cutoff radius |
| Yᵢ^{e} | The ground-truth energy of the | C_{T} | The number (constant) of edges of |
| | i-th atom | | an atomic graph when the atomic |
| | | | graph is a directed complete graph |
| Yᵢ^{f} | The ground-truth force of the i- | m | The number of element types |
| | th atom | | included in a current data set |
| α | The ratio of energy accuracy in | wᵢ | The weight (or mass) of the i-th |
| | requirements | | key sample |
| β | The ratio of force accuracy in | Mᵢ | The relative atomic mass of the i- |
| | requirements | | th atom |

In [Table 1], the predicted energy and predicted force may represent predicted values of energy and force output using a GNN-based potential energy model. In some cases, a ground-truth energy and a ground-truth force may represent actual value data for atoms and may obtained from the data set. For example, the electronic device may determine the actual energy and actual force of the atoms in the data set using the Vienna Ab initio simulation package (VSAP) based on a first-principles modeling simulation of material atoms, though embodiments are not necessarily limited thereto.

According to an embodiment, the electronic device may implement a loss function such as the one described by [Equation 2]. This loss function may be selected or adjusted based on a user input. The electronic device may achieve rapid convergence and stabilization of the loss function and reduce the amount of computation by defining a term for regularization regarding the number of edges of the atomic graph in the balanced loss function of [Equation 2] and a term regarding a weight (or mass) and atomic weight of a sample for stabilizing the loss function.

The electronic device may significantly improve the computational efficiency of the hyperparameter adjustment of the potential energy model by using the balanced loss function above, and achieve small fluctuation and rapid convergence by comprehensively considering an accuracy index (e.g., energy accuracy or force accuracy) and a performance index (e.g., a computational amount index such as the number of edges of the atomic graph or the atomic weight of the key sample) in accordance with [Equation 2].

Referring to FIG. 3, the electronic device may adjust the cutoff radius using a parameter adjuster based on the loss determined according to the loss function. The electronic device may re-execute training of the potential energy model based on the adjusted cutoff radius and the key sample.

According to an embodiment, the parameter adjuster may use a sequential model-based optimization for general algorithm configuration (SMAC) algorithm, which may solve an issue that a parameter type may not be discretely processed in the Gaussian regression process. Unlike conventional gradient descent and backpropagation methods which use differentiable objective functions and can with discrete or categorical hyperparameters, the SMAC algorithm can handle mixed-type parameter spaces including continuous, discrete, and categorical variables. This method may be used to optimize hyperparameters like cutoff radii that may have complex, non-differentiable relationships with model performance. The SMAC algorithm may utilize Gaussian process models for optimization, and a random forest model class may be introduced into a sequential model-based optimization (SMBO) technique to process categorical parameters.

According to an embodiment, the weighting parameters a, B, and y in the loss function of [Equation 2] provide flexible control over the balance between model accuracy and computational efficiency of the potential energy model, thereby improving the training optimization process by allowing adjustment of energy accuracy requirements, force accuracy requirements, and computational load constraints.

According to an embodiment, when training the GNN-based potential energy model, the electronic device may integrate deep learning interatomic potential models with hyperparameter optimization toolkits through code modification and optimization, perform parallel training by modifying the training framework of the potential energy model, and efficiently utilize computational resources. Some embodiments may use toolkits such as NNI (Neural Network Intelligence) to automate hyperparameter tuning and distributed training processes.

According to the hyperparameter determination method as described above, computing performance (e.g., the simulation speed when running the GNN-based potential energy model with various numbers of GPUs) may be greatly improved. In addition, when a simulation of a semiconductor material production scenario is performed, since a selected key sample has the same or similar distribution as the original data set, training using the key sample may achieve substantially the same accuracy as training using the original data set. In addition, the accuracy of a model trained using the key sample may be substantially the same as the accuracy of a model trained using the original data set. Embodiments may utilize support vector regression techniques for key sample selection, thereby significantly shortening the training time while maintaining model accuracy substantially equivalent to training using all samples. Some embodiments may use optimized SVR variants such as widely-interval SVR (NuSVR) to further enhance the efficiency of the key sample selection process.

The cutoff radius, which is determined based on the importance value of each elemental combination of the data set, may effectively reduce the number of times the cutoff radius is adjusted and save resources. In addition, when an environmental descriptor that combines position coordinates with force coordinates is used, the time required to determine an optimal value of the cutoff radius may be significantly shortened. When a loss function such as [Equation 2] is used, an optimal cutoff radius may be determined by weighting the accuracy of a model and the amount of computation, so that the balance between the accuracy of the model (e.g., force accuracy) and the amount of computation may be appropriately adjusted. In conclusion, the GNN-based potential energy model trained through the above-described method may improve the computational performance of an MD simulation of a molecule (e.g., a silicon compound) while ensuring the accuracy of the model.

FIG. 6A illustrates an example of a process for obtaining a key sample, according to an embodiment. FIG. 6B illustrates a flowchart for obtaining a key sample according to an embodiment.

FIG. 6A illustrates original cube-structured unit cell samples 605 and their corresponding sample weights 615. Each cubic structure in the original unit cell sample 605 represents an individual sample (or original sample) containing at least one atom, with different cube types (N1, N2, etc.) representing different atomic compositions or configurations. The weight of the unit cell sample 605 shows the relative importance or frequency of each sample type, expressed as ratios (L1/L, L2/L, L3/L, etc.). The weighted SVR model 625 processes these weighted samples to identify the most representative structures, and the system obtains the selected key sample 635 therefrom, which contains a reduced but statistically representative subset of the original sample types.

According to an embodiment, operations 610 to 630 of FIG. 6B may be performed by an electronic device (e.g., the electronic device 1100 of FIG. 11 or the electronic device 1200 of FIG. 12). The electronic device may include at least one processor including processing circuitry. The electronic device may include at least one memory including one or more storage media including instructions. The instructions, when executed by the at least one processor individually or collectively, may cause the electronic device to perform at least some of the operations related to the training method of the present disclosure.

In the following embodiments, operations may be performed sequentially but not necessarily. For example, the order of the operations may change, and at least two of the operations may be performed in parallel.

According to an embodiment, operation 210 of obtaining the key sample from the plurality of samples of the data set for an MD simulation of FIG. 2 may include obtaining a key sample from the plurality of samples of the data set using a weighted SVR. The weighted SVR model, after training, reveals support vectors (samples) that best represent the underlying data distribution while accounting for the varying importance of the sample types. The operation of obtaining a key sample from the plurality of samples of the data set using the weighted SVR may include operations 610 to 630 of FIG. 6B.

In operation 610, the electronic device may determine a weight of each of the plurality of samples based on statistical characteristics of the plurality of samples of the data set. The weights may reflect the relative importance and representativeness of each sample within the broader dataset, with less common but structurally significant configurations receiving higher weights to ensure they remain adequately represented in the reduced key sample set.

The electronic device may obtain the plurality of samples from the data set (or the original data set). The electronic device may determine each unit cell in the data set as a unit cell sample.

The electronic device may determine the statistical characteristics of the plurality of samples of the data set to guide the weighting process. The statistical characteristics of the samples may include the number (or ratio) of samples having the same or similar characteristics in the data set.

For example, the statistical characteristics of the samples may include statistical characteristics related to the number of atoms, such as the number of unit cell samples having the same number of atoms. Since the number of atoms and the distribution of atoms in each sample of the data set may be different from each other, the ratio of samples having a predetermined number of atoms among all samples included in the data set may be set as a statistical characteristic for measuring the degree of importance of the corresponding samples. However, the statistical characteristics of the samples are not limited to the present disclosure and may be determined in any appropriate manner.

The electronic device may determine the count (or number) of one or more samples having the same or similar number of atoms among the plurality of samples of the data set. The one or more samples having the same (or similar) number of atoms may be referred to as a “sample set (or unit cell sample set).” In other words, the electronic device may determine sample sets having different numbers of atoms among the plurality of samples of the data set.

The electronic device may determine the ratio of the count of one or more samples having the same number of atoms to the total number of plurality of samples as a statistical characteristic. In other words, the electronic device may determine the ratio of the samples included in each sample set among the total samples as a statistical characteristic.

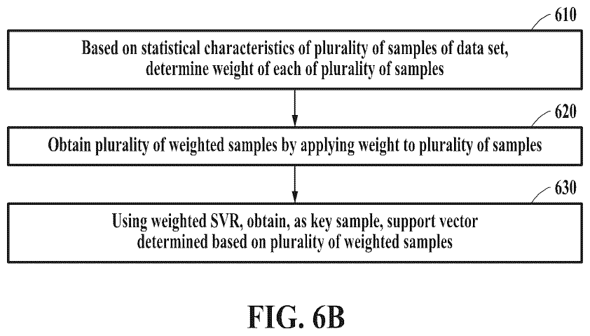

The electronic device may determine a weight of each of the plurality of samples based on the statistical characteristics of the plurality of samples. These calculated ratios correspond to the weight of the unit cell sample 615 shown in FIG. 6A. The electronic device may calculate the ratio of the samples included in each sample set among the total samples as a statistical characteristic. A method of determining weights of samples is described in detail with reference to FIG. 7.

In operation 620, the electronic device may obtain a plurality of weighted samples by applying a weight to the plurality of samples. The electronic device may obtain a plurality of weighted samples by multiplying the weights respectively corresponding to the plurality of samples (or sample items). Although the samples may be weighted by multiplication, the present disclosure is not necessarily limited thereto, and the electronic device may obtain the weighted samples by other weighting techniques.

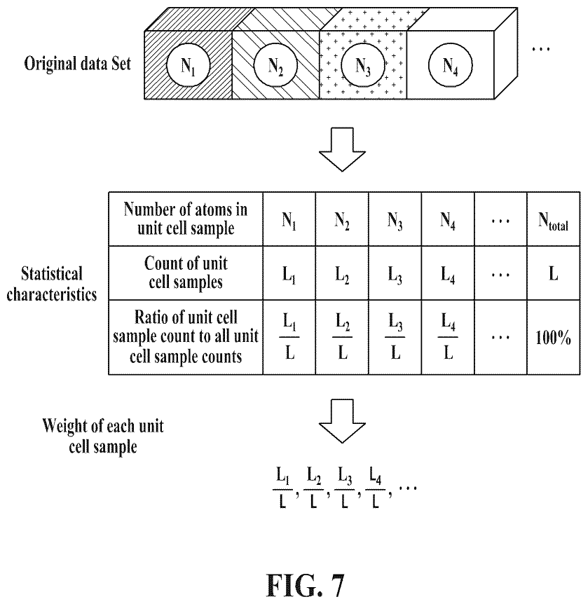

In operation 630, the electronic device may obtain a support vector determined based on the plurality of weighted samples as a key sample using a weighted SVR. A method of obtaining a key sample is described in detail with reference to FIG. 8.

FIG. 7 illustrates an example of a process of determining weights of samples, according to an embodiment. The data set 700 contains the original unit cell samples used for analysis. The electronic device extracts statistical characteristics 705 from the data set 700, which include frequency distributions and structural properties of the unit cell samples. Based on these statistical characteristics 705, the electronic device calculates the weight of each unit cell sample 710, representing the relative importance or representativeness of each sample type within the overall data set.

As described with reference to FIGS. 6A and 6B, an electronic device may obtain a plurality of unit cell samples from a data set (or original data set).

The electronic device may determine a set of unit cell samples having the same or similar number of atoms based on the number of atoms included in each unit cell sample (or original unit cell sample). For example, the original data set may include one or more unit cell samples having N1 atoms, one or more unit cell samples having N2 atoms, one or more unit cell samples having N3 atoms, and one or more unit cell samples having N4 atoms.

The electronic device may determine a set of unit cell samples having N1 atoms, a set of unit cell samples having N2 atoms, a set of unit cell samples having N3 atoms, and a set of unit cell samples having N4 atoms.

The electronic device may determine a count L1 of unit cell samples having N1 atoms, a count L2 of unit cell samples having N2 atoms, a count L3 of unit cell samples having N3 atoms, and a count L4 of unit cell samples having N4 atoms. The electronic device may determine the total number L of the plurality of samples included in the original data set. The electronic device may determine the ratio of the count of one or more unit cell samples having the same number of atoms to the total number L of plurality of samples.

The electronic device may determine a weight of each of the plurality of samples based on the ratio of the count of one or more unit cell samples having the same (or similar) characteristic (e.g., the number of atoms) to the total number of plurality of samples included in the original data set. For example, the electronic device may determine, as the weight of a corresponding unit cell sample, the ratio of the count of one or more unit cell samples having the same (or similar) characteristic (e.g., the number of atoms) to the total number of plurality of samples.

The above description uses unit cell samples having the same number of atoms for clarity. The electronic device may group unit cell samples having similar numbers of atoms when the data set contains samples with highly diverse atom counts. For example, the electronic device can group unit cell samples into the same set when the difference in atom count between samples falls within a defined tolerance range.

FIG. 8 illustrates an example of a method of obtaining a key sample using a weighted support vector regressor (SVR), according to an embodiment.

A support vector regressor (SVR) is a type of support vector machine configured for regression tasks. An SVR seeks to determine a regression function that fits a plurality of input samples within a margin of tolerance, sometimes referred to as an E-insensitive region. The regression function may be expressed as ƒ(x)=w^{T}x+b, where w is a weight vector and b is a bias term. As illustrated in FIG. 8, the E-insensitive region may be defined by an upper boundary ƒ(x)+σ and a lower boundary ƒ(x)−σ, which may be represented as dotted lines parallel to the regression function. According to some aspects, samples located within the ε-insensitive region do not contribute to the definition of the regression function, whereas samples lying on or outside the boundaries of the region, referred to as support vectors, influence the determination of the regression function. In some implementations, the axes of FIG. 8 may correspond to input features (e.g., x₁, x₂, . . . ), and the regression function defines the relationship between such input features and a predicted continuous value.

As described with reference to FIGS. 6A to 7, an electronic device may determine a weight for each of a plurality of samples based on statistical characteristics of the plurality of samples of a data set. The electronic device may obtain a plurality of weighted samples by applying the weights to the plurality of samples.

According to an embodiment, the electronic device may train (or fit) a weighted SVR based on a weighted support vector machine (SVM) using the plurality of weighted samples. Support vector machines are machine learning algorithms that find optimal decision boundaries by identifying support vectors, which are the most informative data points that define the boundary between different classes or regions in the data space. Support vector regression extends this concept to predict continuous values rather than discrete classifications.

Referring to FIG. 8, the SVM may transform the plurality of weighted samples to be linearly separable in a feature space using an appropriate kernel function. The kernel function maps the original data into a higher-dimensional space where complex, non-linear relationships in the original space become linearly separable. Some kernel functions include polynomial, radial basis function (RBF), and sigmoid kernels. The electronic device may perform a regression task to predict a true energy value and a true force value of each weighted sample using the SVR.

In the graph of FIG. 8, ƒ(x) is an analytical expression of a regression hyperplane, which may be defined by an SVM model. In this example, w and b are parameters of the SVM model, where w may represent a weight parameter vector of the SVM model, and b may represent an offset parameter vector of the SVM model.

In the example graph of FIG. 8, the thick solid line may represent the regression hyperplane, the dotted lines may represent soft margins, and the shaded region may represent a o boundary band (sometimes referred to as an epsilon band or an E-band). The samples indicated by the dotted circles may represent samples that are located within the boundary band and are regressed without loss (or correct samples), and the samples indicated by the solid circles may represent samples that touch or exceed the boundaries of the soft margins and cause a loss and are the support vectors. The electronic device may determine a sample that is located near the soft margins or outside the soft margins (e.g., a support vector) as a key sample. In some cases, one or more support vectors are selected.

Since a key sample (or support vector) obtained through SVR fitting are half or less than the samples included in the original data set, the amount of computation using a subsequent key sample may be significantly reduced.

According to some aspects, the structural characteristics of an SVR cause most samples to be excluded from the model after training completion. The final SVM model may be defined only by selected support vectors. Therefore, the sample obtained by sampling the original data set maintains substantially the same structural distribution as the original data set. The training time is shortened and the prediction accuracy is maintained.

The method of obtaining a key sample by sampling an original data set using a weighted SVR is described above, but the method of obtaining a key sample is not limited to the present disclosure, and a key sample may be obtained through other types of sampling techniques to reduce data set sizes.

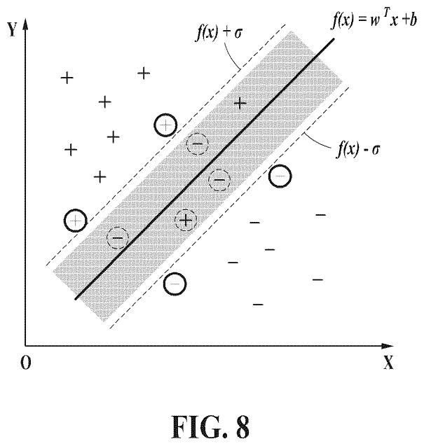

FIG. 9 illustrates changes in a neighborhood of SiN molecules before and after a cutoff radius is adjusted, according to an embodiment.

Semiconductor materials may often include other chemical elements in addition to a silicon (Si) element. For example, referring to FIG. 9, a silicon and nitrogen (SiN) data set may include two types of atoms, silicon (Si) atoms and nitrogen (N) atoms.

(a) of FIG. 9 illustrates an example atomic graph of a potential energy model using a fixed (or preset) cutoff radius. For example, in (a) of FIG. 9, the same fixed cutoff radius (Rₘₐₓ) (e.g., 6.0 nanometers (nm)) may be used for all of the nitrogen-nitrogen (N—N) elemental combinations, nitrogen-silicon (N—Si) elemental combinations, and silicon-silicon (Si—Si) elemental combinations.

A neighboring atom is an atom located within the cutoff radius range of a central atom. The cutoff radius may define the maximum distance at which atomic interactions are calculated. For example, (a) of FIG. 9 shows all neighboring atoms within the cutoff radius of a nitrogen atom as the central atom.

The cutoff radius determines the number of neighboring atoms included in calculations for each central atom. Accordingly, cutoff radius affects both computational load and model accuracy. A larger cutoff radius increases the number of neighboring atoms and computational requirements. A smaller cutoff radius reduces computational load but may exclude relevant atomic interactions.

According to an embodiment, an electronic device may determine different cutoff radii for different elemental combinations when determining a cutoff radius in order to accelerate the process of adjusting the cutoff radius and obtain a better initial value of the cutoff radius. The electronic device may determine each cutoff radius by considering the influence of different elemental combinations on the number of edges of the atomic graph to more appropriately set each cutoff radius. A method of determining a cutoff radius of each elemental combination based on the degree to which each elemental combination contributes to the number of edges of an atomic graph is described with reference to FIG. 10.

FIG. 10 illustrates a flowchart for determining a cutoff radius of each elemental combination, according to an embodiment.

According to an embodiment, operations 1010 and 1020 may be performed by an electronic device (e.g., the electronic device 1100 of FIG. 11 or the electronic device 1200 of FIG. 12). The electronic device may include at least one processor including processing circuitry. The electronic device may include at least one memory including one or more storage media including instructions. When executed by the at least one processor individually or collectively, the instructions may cause the electronic device to perform at least some of the operations related to the training method of the present disclosure.

According to an embodiment, operation 220 of FIG. 2, determining a cutoff radius of a potential energy model, may include operations 1010 and 1020.

In operation 1010, the electronic device may determine the importance corresponding to the degree to which each elemental combination contributes to the number of edges of an atomic graph for each elemental combination of the atoms of a key sample.

According to an embodiment, operation 1010 includes determining, for each elemental combination of the atoms of the key sample, the importance corresponding to the degree to which each elemental combination contributes to the number of edges of the atomic graph may include determining an environmental descriptor of each of the atoms (e.g., an i-th atom) of the key sample, an operation of determining an element ratio of a neighboring atom within a reference cutoff radius of each of the atoms of the key sample, and an operation of determining the importance of each elemental combination of the atoms based on the environmental descriptor and the element ratio of the neighboring atom of each of the atoms.

According to some embodiments, the electronic device can perform the environmental descriptor calculation and the element ratio calculation sequentially or in parallel.

Within each unit cell, an atom might not only have local characteristics, such as physical properties of the atom itself, but a plurality of atoms may have global characteristics, such as spatial characteristics or geometric characteristics. In the present disclosure, an environmental descriptor M of any atom may include information about local characteristics of the atom and global characteristics of a plurality of atoms.

According to an embodiment, the electronic device may calculate an environmental descriptor for each atom based on the atom's position parameter and force parameter, along with the position and force parameters of neighboring atoms within the reference cutoff radius. The electronic device may determine the environmental descriptor M that reflects the local and global characteristics of each of the atoms according to the [Equation 3] below.

$${M}_{i} = {{\sum }_{i = 0 , j \neq i}}^{{N}_{R}} {ω}_{p} {‖ {X}_{i} - {X}_{j} ‖}_{2}^{2} + {ω}_{F} {‖ {F}_{i} - {F}_{j} ‖}_{2}^{2} + {Z}_{i} \qquad ( 3 )$$

In [Equation 3], Mᵢ represents the environmental descriptor of the i-th atom, and N_{R} represents the number of atoms in a divided region using a preset (or fixed) cutoff radius R. The preset cutoff radius R may also be referred to as a “reference cutoff radius.” For example, the reference cutoff radius R may be preset according to a calculation capacity.

Xᵢ represents the position coordinates of the i-th atom, and Xᵢ represents the position coordinates of a j-th atom. Fᵢ represents the force coordinates of the i-th atom, and Fⱼ represents the force coordinates of the j-th atom. The j-th atom may be a neighboring atom (or an atom other than the i-th atom within the reference cutoff radius R with the i-th atom as the central atom) in the divided region using the reference cutoff radius R with the i-th atom as the central atom.

Wp represents the weight of the position coordinates of an atom, and wp represents the weight of the force coordinates of the atom. Op and we may be set according to an actual MD simulation environment. For example, in a simulation environment that satisfies a periodic boundary condition, ωₚ and ω_{F} may be set to 1:1. The periodic boundary condition may represent a condition for maintaining the number of particles in an MD simulation system constant during the movement of the particles.

Zᵢ is an energy transfer parameter and may be an offset of the coordinates of the displacement under the periodic boundary condition. Zᵢ may be used optionally, and it's usage may be determined depending on whether the periodic boundary condition is satisfied. For example, when the periodic boundary condition is not satisfied, Zᵢ may not be used. For example, when the periodic boundary condition is satisfied, Zᵢ may be used.

According to some aspects, these parameters are learned during model training and stored in a non-transitory memory of the electronic device as part of the trained potential energy model configuration. The parameters (e.g., Fᵢ or Fⱼ) representing the force coordinates may reflect the local characteristics of the atom. The parameters (e.g., Xᵢ or Xⱼ) representing the position coordinates may represent the distribution of the atomic data in a data set by reflecting global characteristics such as spatial characteristics or geometric characteristics of the atoms in a unit cell. An environmental descriptor determined according to [Equation 3] may be used to determine the importance value, which represents the degree to which each elemental combination contributes to the number of edges in the atomic graph. The importance of each elemental combination may be used to evaluate the influence (or sensitivity) of corresponding elemental combination on the number of edges in the atomic graph.

The element ratio of a neighboring atom may represent the ratio of an element of the neighboring atom among all elements within the neighborhood range of the central atom. According to an embodiment, determining the element ratio of the neighboring atom within the reference cutoff radius of each of the atoms of the key sample may include determining the number of neighboring atoms corresponding to each element within the reference cutoff radius of each of the atoms of the key sample, an operation of determining the total number of neighboring atoms within the reference cutoff radius of each of the atoms, and an operation of determining, as the element ratio of a neighboring atom corresponding to each element, the ratio of the number of neighboring atoms corresponding to each element to the total number of neighboring atoms within the reference cutoff radius of each of the atoms.

For example, a method of determining the importance value of each elemental combination for cutoff radii of three elemental combinations of nitrogen-nitrogen, nitrogen-silicon, and silicon-silicon is described below. This method may correspond to operation 220 of FIG. 2 and block 220 of FIG. 5, which includes determining an importance value after determining environmental descriptor. The cutoff radius of the nitrogen-nitrogen elemental combination may be represented as R_N_N, and the cutoff radius of the nitrogen-silicon elemental combination may be represented as R_N_Si.

Referring to (a) of FIG. 9, in an example where a nitrogen atom is the central atom, and the number (C_{N}) of neighboring atoms whose element type is nitrogen of the nitrogen atom is 25 and the number (C_{Si}) of neighboring atoms whose element type is silicon of the nitrogen atom is 36, the total number (Cₜₒₜₐₗ) of neighboring atoms of the nitrogen atom is 25+36=61. The element ratio (Ratio_{N}) of a nitrogen neighboring atom (or nitrogen-nitrogen elemental combination) of the nitrogen atom may be determined as 25/61≈40.98%, which is the ratio of the number (C_{N}) of nitrogen neighboring atoms to the total number (Cₜₒₜₐₗ) of neighboring atoms. The element ratio (Ratio_{Si}) of a silicon neighboring atom (or nitrogen-silicon elemental combination) of the nitrogen atom may be determined as 36/61˜59.02%, which is the ratio of the number (C_{Si}) of silicon neighboring atoms to the total number (Cₜₒₜₐₗ) of neighboring atoms.

After determining the environmental descriptor and the element ratio of the neighboring atom of each of the atoms of the key sample, the electronic device may determine an importance value corresponding to the degree to which each elemental combination contributes to the number of edges of the atomic graph. The electronic device may determine the importance value of each elemental combination through the processing described in [Table 2].

The electronic device may obtain data (or input data) including parameters (e.g., position coordinates, an initial energy value, force coordinates, and an energy offset) at least corresponding to the “input” of [Table 2] based on the original data set. For example, the electronic device may obtain input data including corresponding parameters from the original data set using the Vienna Ab initio Simulation Package (VASP).

TABLE 2

| | |
|---|---|
| Input | Position coordinates, initial energy values, force coordinates, and energy |
| | offsets of atoms within a unit cell sample |
| Output | The importance value corresponding to the degree to which each elemental |
| | combination contributes to the number of edges in an atomic graph: |
| | Importance(element, element2) |
| | (“element” may represent the first element of each elemental combination, and |
| | “element2” may represent the second element that may be the same as or |
| | different from the first element of a corresponding elemental combination) |
| Process | (1) The electronic device may execute the following operations for the i-th atom |
| detail | among all atoms in one unit cell sample of the original data set (or key sample). |
| | The electronic device may repeatedly execute the following operations for each |
| | of all atoms in a unit cell sample. |
| | (2) The electronic device may calculate the environmental descriptor (Mᵢ) of |
| | the i-th atom according to the reference cutoff radius R and [Equation 3]. |
| | (3) The electronic device may calculate the number of neighboring atoms (e.g., |
| | C₁, C₂, C₃, . . . Cₘ) of different element types among all neighboring atoms of the |
| | i-th atom in the neighborhood of the i-th atom. |
| | The subscript of C may be used to distinguish different neighboring atoms of |
| | different element types. For example, C₁ may represent the number of nitrogen |
| | neighboring atoms. m may represent the maximum number of element types |
| | currently included in the original data set. |
| | (4) The electronic device may determine an element ratio by dividing the |
| | number of neighboring atoms (C₁, C₂, C₃, . . . Cₘ) of each element type in the |
| | neighborhood of the i-th atom by the number of all neighboring atoms of the i- |
| | th atom. |
| | (5) The electronic device may sort all atoms in a unit cell sample according to |
| | the order of the sizes of the environmental descriptors of the corresponding |
| | atoms. For example, the electronic device may sort all atoms in the unit cell |
| | sample according to the decreasing order of the sizes of the environmental |
| | descriptors of all atoms. |
| | (6) The electronic device may select K atoms corresponding to the first K |
| | environmental descriptors. For example, the electronic device may select K |
| | atoms corresponding to the K largest environmental descriptors in the unit cell |
| | sample. |
| | Particularly, the electronic device may select K atoms that are more important |
| | among all atoms in the unit cell sample. K may be a preset hyperparameter |
| | (e.g., 3000) and may be adjusted according to the ratio of the atoms in the unit |
| | cell sample. The K atoms may have the same or different element types. |
| | (7) The electronic device may determine the average value of the element ratios |
| | of respective elemental combinations of K atoms. For example, the electronic |
| | device may determine the sum of the element ratios (Ratio_{N}) of the nitrogen |
| | neighboring atoms of all nitrogen atoms (or nitrogen central atoms) among K |
| | atoms for the nitrogen-nitrogen elemental combination and determine the |
| | corresponding average value. |
| | (8) The electronic device may repeatedly perform operations (1) to (7) |
| | described above for all unit cell samples of the original data set (or key sample). |
| | (9) The electronic device may determine (or return) the average value of the |
| | element ratios of respective the elemental combinations as the importance value |
| | of each elemental combination of the original data set (or key sample). |

According to an embodiment, determining the importance value of each elemental combination of the atoms based on an environmental descriptor and the element ratio of the neighboring atom of each of the atoms of the key sample may include determining a determined number of atoms among the atoms of a first sample based on environmental descriptors of atoms of a first sample among a plurality of samples of the key sample and an operation of determining, based on the element ratio of the neighboring atoms of a second element type for each of one or more atoms of a first element type among the determined number of atoms of the first sample, the importance value of elemental combination corresponding to the first element type and the second element type. The second element type may be the same as or different from the first element type.

For example, the operation of determining, based on the element ratio of the neighboring atoms of the second element type for each of one or more atoms of the first element type, the importance value of the elemental combination corresponding to the first element type and the second element type may include determining the average value of the element ratio of the neighboring atoms of the second element type for each of one or more atoms of the first element type as the importance value of the elemental combination corresponding to the first element type and the second element type.

For example, the first element type may be nitrogen, and the second element type may be nitrogen. The electronic device may determine the importance of the nitrogen-nitrogen elemental combination based on the element ratio of a nitrogen neighboring atom to each of the nitrogen atoms among the determined number of atoms of the first sample. The electronic device may determine, as the importance value of the nitrogen-nitrogen elemental combination, the average of the element ratio of the nitrogen neighboring atoms for each of the nitrogen atoms among the determined number of atoms of the first sample. The electronic device may determine the importance value of each of all elemental combinations of the key sample. The above operations described with reference to [Table 2] may be performed by an electronic device including a processor and a memory, the memory storing instructions executable to calculate environmental descriptors for atoms in unit cell samples, determine element ratios of neighboring atoms within reference cutoff radii, sort atoms based on environmental descriptor values, select a predetermined number of atoms with largest environmental descriptors, calculate average element ratios for elemental combinations across selected atoms, and output importance values corresponding to each elemental combination's contribution to atomic graph edge count.

Referring to [Table 3], examples of the environmental descriptor and element ratio of the SiN original data set are described. For example, the data in [Table 3] may be calculated through the processing as described in [Table 2].

TABLE 3

| | | Element | | | The element | The element |
|---|---|---|---|---|---|---|
| | | type of | The number | The number | ratio of a | ratio of a |
| Central atom | | the | of nitrogen | of silicon | nitrogen | silicon |
| i in the | Environmental | central | neighboring | neighboring | neighboring | neighboring |
| first sample | descriptor Mᵢ | atom | atoms C_{N} | atoms C_{Si} | atom Ratio_{N} | atom Ratio_{Si} |
| 1 | M₁ | N | 25 | 36 | 40.98% | 59.02% |
| 2 | M₂ | N | 14 | 27 | 34.15% | 65.85% |
| 3 | M₃ | Si | 57 | 68 | 45.60% | 54.40% |
| . . . | . . . | . . . | . . . | . . . | . . . | . . . |

[Table 3] shows the environmental descriptor (Mᵢ) of the i-th atom and the element ratio of a neighboring atom of the i-th atom (i=1, 2, 3, . . . ).

For example, a first (i=1) atom may be a nitrogen atom (or an atom whose element type is nitrogen), the environmental descriptor of the first atom may be M₁, the element ratio (Ratio_{N}) of a nitrogen neighboring atom (or an atom whose element type is nitrogen) of the first atom may be 40.98%, and the element ratio (Ratio_{Si}) of a silicon neighboring atom (or an atom whose element type is silicon) may be 59.02%.

A second (i=2) atom may be a nitrogen atom, the environmental descriptor of the second atom may be M₂, the element ratio (Ratio_{N}) of a nitrogen neighboring atom of the second atom may be 34.15%, and the element ratio (Ratio_{Si}) of a silicon neighboring atom may be 65.85%.

A third (i=3) atom may be a silicon atom, the environmental descriptor of the third atom may be M₃, the element ratio (Ratio_{N}) of a nitrogen neighboring atom of the third atom may be 45.60%, and the element ratio (Ratio_{Si}) of a silicon neighboring atoms may be 54.40%.

For example, when K is 3000, the electronic device may select 3000 atoms from all the atoms in the first sample based on an environmental descriptor (e.g., M₁, M₂, M₃, etc.). The electronic device may determine the importance value of each elemental combination of the atoms in the first sample according to [Equation 4].

$${Importance}_{( N , N )} = mean ( {{\sum }_{i = 1}}^{k} {Ratio}_{N} ) \qquad [ Equation 4 ]$$

$${Importance}_{( N , Si )} = mean ( {{\sum }_{i = 1}}^{k} {Ratio}_{Si} )$$

$${Importance}_{( Si , Si )} = mean ( {{\sum }_{i = 1}}^{k} {Ratio}_{Si} )$$

Importance (N,N) represents the importance value of the nitrogen-nitrogen (N—N) elemental combination, Importance_{(N,Si)} represents the importance of the nitrogen-silicon (N—Si) elemental combination, and Importance_{(Si,Si)} represents the importance of the silicon-silicon (Si—Si) elemental combination. ‘k’ represents any number of atoms among K atoms, and k may be less than or equal to K.

For example, for the N—N elemental combination, since silicon atoms among the current K atoms might not form the N—N elemental combination, in this case, k is a value obtained by subtracting the number of silicon atoms among the K atoms from K and i represents the i-th atom among the k atoms. For example, when calculating the importance value of an arbitrary elemental combination, the electronic device might not consider atoms that do not belong to that elemental combination.

The electronic device may determine, for k atoms among K atoms, the average value of element ratio of neighboring atoms of the second element type for each of one or more atoms of the first element type as the importance value of the elemental combinations corresponding to the first element type and the second element type. The electronic device may perform the above-described operation for each elemental combination.

The importance value of each elemental combination may be used to evaluate the influence (or sensitivity) of a corresponding elemental combination on the number of edges of the atomic graph. The electronic device may determine a hyperparameter for training a potential energy model based on the importance value.

Referring again to FIG. 10, in operation 1020, the electronic device may determine a cutoff radius of each elemental combination of the atoms based on the importance value of each elemental combination of the atoms.

According to an embodiment, operation 1020 of determining a cutoff radius based on the importance value of each elemental combination of the atoms may include determining a cutoff radius of each elemental combination within a defined cutoff radius range based on the importance value of each elemental combination of the atoms. The electronic device may sort the elemental combinations based on the importance value (or the order of importance by magnitude) of each elemental combination.

The greater the importance value of any elemental combination, the greater the influence of that elemental combination on the number of edges in the atomic graph. In other words, the greater the importance value of any elemental combination, the more sensitive the atoms belonging to that elemental combination are to neighboring atoms, and as a result, that elemental combination may be more sensitive to the amount of computation. For example, in the SiN data set, the electronic device may determine different cutoff radii for different elemental combinations by considering the relative importance of respective elemental combinations, when the importance value (e.g., 0.2114) of the N—N elemental combination<the importance value (e.g., 0.2561) of the Si—Si elemental combination<the importance value (e.g., 0.6232) of the N—Si elemental combination.

This element-specific cutoff radius determination addresses limitations of fixed cutoff radius methods used in conventional MD simulation systems. Fixed cutoff radius methods, which apply the same radius value to all elemental combinations, result in computational inefficiency and suboptimal accuracy. The electronic device of the present embodiments, in contrast, uses different cutoff radii for different elemental combinations based on calculated importance values. This selective approach reduces unnecessary edge calculations for less important elemental combinations while maintaining sufficient interaction coverage for more sensitive combinations. For example, in the SiN data set, the N—Si elemental combination receives a larger cutoff radius due to its higher importance value (0.6232), while the N-N elemental combination uses a smaller radius corresponding to its lower importance (0.2114). This targeted optimization reduces the total number of atomic graph edges while preserving prediction accuracy for energy and force calculations.

In some embodiments, the electronic device or a user may preset or define a cutoff radius range. For example, the electronic device may determine the cutoff radius range as 4 nm to 7 nm according to a requirement for performing an MD simulation. As a cutoff radius increases, the number of neighboring atoms increases. Therefore, the number of edges of an atomic graph may increase, and the amount of computation may increase.

According to an embodiment, the electronic device may determine the cutoff radius of respective elemental combinations within the defined cutoff radius range based on the importance value (e.g., the order of importance-based size) of the elemental combinations. The electronic device may sort elemental combinations in ascending or descending order of importance value based on the importance of each elemental combination.

In some embodiments, the electronic device may assign cutoff radii inversely proportional to elemental combination importance values within a defined cutoff radius range. For example, the electronic device may assign the maximum cutoff radius to the elemental combination with the lowest importance. The electronic device may assign smaller cutoff radii to elemental combinations with higher importance values. In one example, the electronic device electronic device may assign the minimum cutoff radius to the elemental combination with the highest importance. However, this is only an example, and the electronic device electronic device may incorporate additional parameters beyond importance when calculating cutoff radius assignments.

(b) of FIG. 9 illustrates an atomic graph using different cutoff radii. For example, the cutoff radius (R_N_N) of the N—N elemental combination may be 5.5 nm, the cutoff radius (R_N_Si) of the N—Si elemental combination may be 6.0 nm, and the cutoff radius (R_Si_Si) of the Si-Si elemental combination may be 5.5 nm (R_Si_Si is not depicted in FIG. 9).

Compared to a divided region using the fixed cutoff radius of (a) of FIG. 9, a region divided according to the cutoff radius determined in the same manner as described with reference to FIGS. 5 to 8 may include a reduced number of nitrogen atoms. A nitrogen atom farther away than the adjusted cutoff radius may be excluded from the divided region. Accordingly, the number of neighboring atoms of the central atom may also be reduced. By adaptively adjusting the cutoff radius of each of the different elemental combinations, the determined cutoff radius may be closer to an optimal cutoff radius value of the potential energy model, and the influence of each elemental combination on the number of edges of the atomic graph may be balanced.

FIG. 11 illustrates a block diagram of an electronic device according to an embodiment.

The electronic device 1100 according to an embodiment may include at least one processor (hereinafter, processor) 1110 including processing circuitry and a memory 1120 including one or more storage media storing instructions. The instructions, when executed by the processor 1110 individually or collectively, may cause the electronic device 1100 to perform at least some of the operations described with reference to FIGS. 1 to 13 of the present disclosure.

In some embodiments, the electronic device 1100 further includes a communicator connected to the processor 1110 and the memory 1120 to transmit and receive data. The communicator may be connected to another external device to transmit and receive data. Hereinafter, transmitting and receiving “A” may refer to transmitting and receiving “information or data indicating A.”

The communicator may be implemented as circuitry within the electronic device 1100. For example, the communicator may include an internal bus and an external bus. In another example, the communicator may be an element that connects the electronic device 1100 to an external device. The communicator may be an interface. The communicator may receive data from the external device and transmit the data to the processor 1110 and the memory 1120.

The processor 1110 may process the data received by the communicator and/or the data stored in the memory 1120. The “processor” may be a data processing device implemented by hardware including circuitry having a physical structure to perform desired operations. For example, the desired operations may include code or instructions included in a program. For example, the hardware-implemented data processing device may include a microprocessor, a central processing unit (CPU), a GPU, a processor core, a multi-core processor, a multiprocessor, an application-specific integrated circuit (ASIC), or a field-programmable gate array (FPGA).

The processor 1110 may control other components (e.g., a hardware or software component) of the electronic device 1100 and perform various types of data processing or operations. As at least a part of the data processing or operations, the processor 1110 may store commands or data received from other components (e.g., a communicator) in at least a part of the memory 1120, process the commands or data stored in the memory 1120, and store result data in the memory 1120. The operations performed by the processor 1110 may be substantially the same as the operations of the electronic device 1100.

The memory 1120 may store information executable by the processor 1110 to perform a processing operation, such as the operations described herein. The memory 1120 (or one or more storage media included in the memory 1120) may store instructions executed by the processor 1110 and store related information while software or a program is executed in the electronic device 1100. For example, the memory 1120 may include one or more memories, which are volatile and/or non-volatile memories known in the field, such as random-access memory (RAM), dynamic RAM (DRAM), static RAM (SRAM), non-volatile RAM (NVRAM), persistent memory (PMEM), magneto-resistive RAM (MRAM), high bandwidth memory (HBM), or 3DXPoint.

The electronic device 1100 may be connected to an external memory through a communicator. For example, the external memory may include one or more volatile memories, non-volatile memories and RAM, flash memory, hard disk drives, and optical disc drives. The external memory may store a set of instructions (e.g., software) for operating the electronic device 1100. The set of instructions for operating the electronic device 1100 may be executed by the processor 1110.

For example, the processor 1110 may obtain a key sample from a plurality of samples of a data set for an MD simulation. The processor 1110 may determine a cutoff radius of a potential energy model based on the key sample. The processor 1110 may determine the cutoff radius by calculating importance values for elemental combinations using environmental descriptors and element ratios. The processor 1110 may execute training of the potential energy model using a first atomic graph generated based on the key sample and the cutoff radius. The processor 1110 may determine an adjusted cutoff radius based on an inference result of the potential energy model trained using the first atomic graph. The processor 1110 may perform one or more training iterations until a set of criteria is reached, such as maximum computation allotment or accuracy thresholds, as described with reference to FIGS. 1, 3, and 5. The processor 1110 may update parameters using SMAC optimization during the iterative training process as described with reference to FIG. 3.

FIG. 12 illustrates a block diagram of an electronic device configured to determine a hyperparameter of a GNN-based potential energy model according to an embodiment.

Referring to FIG. 12, the electronic device 1200 may include a key sample obtaining module 1210, a hyperparameter initial value determination module 1220, and a hyperparameter adjustment module 1230. Each module may correspond to specific processing stages in the adaptive cutoff radius optimization workflow. The modules may be implemented as dedicated processors, application specific integrated circuits (ASICs), or a set of instructions executable by a processor as described with reference to FIG. 11.

The electronic device 1200 may include at least some components of an electronic device 1000 described with reference to FIG. 10. For example, the electronic device 1200 may include at least one processor 1110. The electronic device 1200 may include a memory 1120. The memory 1120 stores instructions executable by the processor 1110 to implement the module functions described below.

The key sample obtaining module 1210 may be configured to obtain a key sample from a plurality of samples of a data set for an MD simulation. For example, the electronic device 1200 may perform operation 210 described with reference to FIG. 2 through the key sample obtaining module 1210. Embodiments of the key sample obtaining module 1210 implement weighted SVR processing to identify support vectors that represent the original dataset's structural distribution.

The hyperparameter initial value determination module 1220 may be configured to determine a cutoff radius of a potential energy model based on a key sample. The cutoff radius may include a cutoff radius of each of elemental combinations. For example, the electronic device 1200 may perform operation 220 described with reference to FIG. 2 through the hyperparameter initial value determination module 1220. The hyperparameter initial value determination module 1220 may calculate importance values for elemental combinations using environmental descriptors and element ratios as described with reference to Table 2.

The hyperparameter adjustment module 1230 may be configured to execute training of the potential energy model based on the key sample and the cutoff radius and adjust the cutoff radius based on an inference result of the trained potential energy model. An input to the potential energy model may include an atomic graph (e.g., a first atomic graph) determined based on the key sample and the cutoff radius. An output of the potential energy model may include an energy parameter and a force parameter of an atom. For example, the electronic device 1200 may perform operations 230 and 240 described with reference to FIG. 2 via the hyperparameter adjustment module 1230. The hyperparameter adjustment module 1230 may use SMAC optimization to update cutoff radius values based on loss function feedback until convergence criteria are satisfied.

According to an embodiment, the key sample obtaining module 1210 may be configured to obtain (or select) a key sample from a plurality of samples of a data set using a weighted SVR. Each of the plurality of samples may include at least one atom.

According to an embodiment, the key sample obtaining module 1210 may be configured to determine a weight corresponding to each of the plurality of samples based on statistical characteristics of the plurality of samples of the data set. The key sample obtaining module 1210 may be configured to obtain a plurality of weighted samples by applying weights to the plurality of samples. The key sample obtaining module 1210 may be configured to obtain a support vector determined based on the plurality of weighted samples as a key sample using the weighted SVR. For example, the weighted SVR may assign higher weights to structurally significant but less frequent sample configurations to maintain dataset representativeness.

According to an embodiment, for each elemental combination of the atoms of the key sample, the hyperparameter initial value determination module 1220 may be configured to determine the importance value corresponding to the degree to which each elemental combination contributes to the number of edges of the atomic graph. The hyperparameter initial value determination module 1220 may be configured to determine a cutoff radius of each elemental combination of the atoms based on the importance value of each elemental combination of the atoms.

According to an embodiment, the hyperparameter initial value determination module 1220 may be configured to determine an environmental descriptor of each of the atoms of the key sample and the element ratio of a neighboring atom within a reference cutoff radius of each of the atoms. The hyperparameter initial value determination module 1220 may be configured to determine the importance of each elemental combination of the atoms based on the environmental descriptor and the element ratio of the neighboring atom of each of the atoms.

According to an embodiment, the hyperparameter initial value determination module 1220 may be configured to determine the environmental descriptor of each of the atoms based on a position parameter and a force parameter of each of the atoms of the key sample and a neighboring atom within the reference cutoff radius of each of the atoms.

According to an embodiment, the hyperparameter initial value determination module 1220 may be configured to determine a cutoff radius of each elemental combination within a defined cutoff radius range based on the importance of each elemental combination of the atoms. The hyperparameter adjustment module 1230 may be configured to determine a loss based on the inference result, the cutoff radius, and the key sample. The hyperparameter adjustment module 1230 may be configured to determine an adjusted cutoff radius based on the loss.

It should be understood that a module (e.g., the key sample obtaining module 1210, the hyperparameter initial value determination module 1220, or the hyperparameter adjustment module 1230) of the electronic device 1200 may be implemented as a hardware component and/or a software component. Those skilled in the art may implement each module using, for example, an FPGA or an ASIC, depending on processing performed by each defined module.

The embodiments described herein may be implemented using a hardware component, a software component and/or a combination thereof. For example, the device, the method, and the components described in the embodiments may be implemented using a general-purpose or special-purpose computer, such as a processor, a controller and an arithmetic logic unit (ALU), a digital signal processor (DSP), a microcomputer, an FPGA, a programmable logic unit (PLU), a microprocessor, or any other devices capable of responding to and executing instructions. A processing device may run an operating system (OS) and one or more software applications that run on the OS. The processing device also may access, store, manipulate, process, and generate data in response to execution of the software. For purpose of simplicity, the description of a processing device is used as singular; however, one skilled in the art will appreciate that a processing device may include a plurality of processing elements and/or a plurality of types of processing elements. For example, the processing device may include a plurality of processors or a single processor and a single controller. In addition, different processing configurations are possible, such as parallel processors.

The software may include a computer program, a piece of code, an instruction, or one or more combinations thereof, to independently or collectively instruct or configure the processing device to operate as desired. Software and data may be embodied permanently or temporarily in any type of machine, component, physical or virtual equipment, or computer storage medium or device capable of providing instructions or data to or being interpreted by the processing device. The software also may be distributed over network-coupled computer systems so that the software is stored and executed in a distributed fashion. The software and data may be stored in a non-transitory computer-readable storage medium.

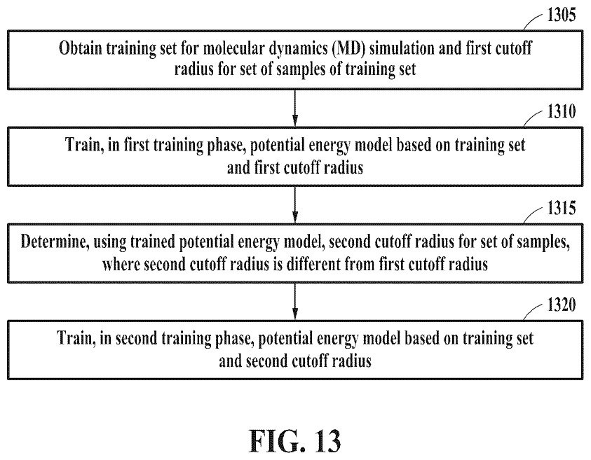

FIG. 13 shows an example of a method 1300 for machine learning according to aspects of the present disclosure. In some examples, these operations are performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations.

At operation 1305, the system obtains a training set for a molecular dynamics (MD) simulation and a first cutoff radius for a set of samples of the training set. In an example, the system extracts unit cell samples from the training set and applies weighted SVR to identify key samples that represent the structural distribution of the original dataset. The system calculates initial cutoff radius values based on element-specific importance values derived from environmental descriptors and element ratios of the key samples. In some cases, the operations of this step refer to, or may be performed by, an electronic device as described with reference to FIG. 11 or FIG. 12.

At operation 1310, the system trains, in a first training phase, a potential energy model based on the training set and the first cutoff radius. In an example, the system generates atomic graphs using the key samples and the first cutoff radius values to define neighbor relationships between atoms. The system executes GNN-based training using the atomic graphs to predict energy and force parameters for molecular configurations. In some cases, the operations of this step refer to, or may be performed by, an electronic device as described with reference to FIG. 11 or FIG. 12.

At operation 1315, the system determines, using the trained potential energy model, a second cutoff radius for the set of samples, where the second cutoff radius is different from the first cutoff radius. In one example, the system evaluates the trained model against defined criteria including energy accuracy, force accuracy, and computational load metrics. The system calculates a loss function that balances prediction accuracy with edge count in the atomic graph and uses SMAC optimization to adjust cutoff radius values based on the loss feedback. In some cases, the operations of this step refer to, or may be performed by, an electronic device as described with reference to FIG. 11 or FIG. 12.

At operation 1320, the system trains, in a second training phase, the potential energy model based on the training set and the second cutoff radius. In an example, the system regenerates atomic graphs using the adjusted cutoff radius values and executes additional training iterations to refine energy and force predictions. The system repeats the training and adjustment cycle until convergence criteria are satisfied or maximum iteration limits are reached. In some cases, the operations of this step refer to, or may be performed by, an electronic device as described with reference to FIG. 11 or FIG. 12.

The method according to the embodiments described above may be recorded in the computer-readable storage medium including program instructions to implement various operations of the embodiments described above. The non-transitory computer-readable storage medium may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as compact disc read-only memory (CD-ROM) discs and digital video discs (DVDs); magneto-optical media such as optical discs; and hardware devices that are specifically configured to store and perform program instructions, such as ROM, RAM, flash memory, and the like. Examples of program instructions include both machine code, such as produced by a compiler, and files containing higher-level code that may be executed by the computer using an interpreter.

The hardware devices described above may be configured to act as one or more software modules in order to perform the operations of the embodiments described above, or vice versa.

As used herein, “A or B,” “at least one of A and B,” “at least one of A or B,” “A, B or C,” “at least one of A, B and C,” and “at least one of A, B, or C,” each of which may include any one of the items listed together in the corresponding one of the phrases, or all possible combinations thereof.

As described above, although the embodiments have been described with reference to the limited drawings, one of ordinary skill in the art may apply various technical modifications and variations based thereon. For example, suitable results may be achieved if the described techniques are performed in a different order, and/or if components in a described system, architecture, device, or circuit are combined in a different manner, or replaced or supplemented by other components or their equivalents.

Therefore, other implementations, other embodiments, and equivalents to the claims are also within the scope of the following claims.

What is claimed is:

1. An electronic device comprising:
at least one processor comprising processing circuitry; and
a memory comprising one or more storage media storing instructions,
wherein the instructions, when executed by the at least one processor individually or collectively, cause the electronic device to perform:
obtaining a key sample from a plurality of samples of a data set for a molecular dynamics (MD) simulation;
based on the key sample, determining a first cutoff radius of a potential energy model;
executing, using a first atomic graph generated based on the key sample and the first cutoff radius, training of the potential energy model; and
determining a second cutoff radius different from the first cutoff radius based on an inference result of the potential energy model trained using the first atomic graph, wherein the inference result of the potential energy model comprises an energy parameter and a force parameter of an atom.

2. The electronic device of claim 1, wherein the obtaining of the key sample comprises:
obtaining, using a weighted support vector regressor (SVR), the key sample from the plurality of samples of the data set.

3. The electronic device of claim 2, wherein:
each of the plurality of samples comprises one or more atoms, and
the obtaining of the key sample comprises:
determining, based on statistical characteristics of the plurality of samples of the data set, a weight corresponding to each of the plurality of samples;
obtaining a plurality of weighted samples by applying the weight to each of the plurality of samples; and
obtaining, as the key sample, a support vector from the plurality of weighted samples using the weighted SVR.

4. The electronic device of claim 3, wherein the determining of the weight corresponding to each of the plurality of samples comprises:
determining a count of one or more samples having a same or similar number of atoms among the plurality of samples of the data set;
computing, as the statistical characteristics, a ratio by dividing the count of the one or more samples having the same or similar number of atoms by a total number of the plurality of samples; and
determining, based on the ratio of the count of the one or more samples to the total number of the plurality of samples, the weight corresponding to each of the plurality of samples.

5. The electronic device of claim 1, wherein the determining of the first cutoff radius of the potential energy model comprises:
determining, for each elemental combination of atoms of the key sample, an importance value corresponding to a degree to which each elemental combination contributes to a number of edges of an atomic graph; and
determining, based on the importance value of each elemental combination of the atoms, the first cutoff radius of each elemental combination of the atoms.

6. The electronic device of claim 5, wherein the determining of the importance value comprises:
determining an environmental descriptor of each of the atoms of the key sample;
computing an element ratio of a neighboring atom within a reference cutoff radius of each of the atoms of the key sample; and
determining, based on the environmental descriptor and the element ratio of the neighboring atom of each of the atoms, the importance value of each elemental combination of the atoms.

7. The electronic device of claim 6, wherein the determining of the environmental descriptor of each of the atoms of the key sample comprises:
generating the environmental descriptor for each atom based on position coordinates and force coordinates of the respective atom and position coordinates and force coordinates of neighboring atoms within the reference cutoff radius of the respective atom.

8. The electronic device of claim 6, wherein the computing of the element ratio of the neighboring atoms within the reference cutoff radius of each of the atoms of the key sample comprises:
determining a number of neighboring atoms corresponding to each element within the reference cutoff radius of each of the atoms of the key sample;
determining a total number of the neighboring atoms within the reference cutoff radius of each of the atoms; and
computing, as the element ratio of the neighboring atom corresponding to the each element, a ratio of the number of neighboring atoms corresponding to the each element to the total number of the neighboring atoms within the reference cutoff radius of each of the atoms.

9. The electronic device of claim 6, wherein the determining of the importance value of each elemental combination of the atoms, based on the environmental descriptor and the element ratio of the neighboring atom of each of the atoms comprises:
determining, based on environmental descriptors of atoms of a first sample of a plurality of samples of the key sample, a defined number of atoms among the atoms of the first sample; and
determining, based on an element ratio of a neighboring atom of a second element type to each of one or more atoms of a first element type among the defined number of atoms of the first sample, the importance value of an elemental combination corresponding to the first element type and the second element type, wherein the second element type is same as or different from the first element type.

10. The electronic device of claim 5, wherein the determining of the cutoff radius of each elemental combination of the atoms comprises determining, based on the importance value of each elemental combination of the atoms, the cutoff radius of each elemental combination within a defined cutoff radius range.

11. The electronic device of claim 1, wherein the determining of the second cutoff radius based on the inference result of the trained potential energy model comprises:
based on the inference result, the first cutoff radius, and the key sample, determining a loss; and
based on the loss, determining the second cutoff radius.

12. The electronic device of claim 1, wherein the instructions, when executed by the at least one processor individually or collectively, cause the electronic device to further perform operations comprising:
generating, based on the second cutoff radius, a second atomic graph as an input to the trained potential energy model;
finetuning, using the second atomic graph, the trained potential energy model; and
obtaining a final potential energy model by repeatedly updating the second cutoff radius, generating a second atomic graph based on the updated second cutoff radius, and finetuning the trained potential energy model until an inference result of the potential energy model satisfies a defined requirement for an MD simulation.

13. A method performed by an electronic device, the method comprising:
obtaining a key sample from a plurality of samples of a data set for a molecular dynamics (MD) simulation;
based on the key sample, determining a first cutoff radius of a potential energy model;
executing, using a first atomic graph generated based on the key sample and the first cutoff radius, training of the potential energy model; and
determining a second cutoff radius different from the first cutoff radius based on an inference result of the potential energy model trained using the first atomic graph, wherein the inference result of the potential energy model comprises an energy parameter and a force parameter of an atom.

14. The method of claim 13, wherein the obtaining of the key sample comprises:
determining, based on statistical characteristics of the plurality of samples of the data set, a weight corresponding to each of the plurality of samples;
obtaining a plurality of weighted samples by applying the weight to each of the plurality of samples; and
obtaining, as the key sample, a support vector from the plurality of weighted samples using a weighted support vector regressor (SVR).

15. The method of claim 14, wherein the determining of the weight corresponding to each of the plurality of samples comprises:
determining a count of one or more samples having a same or similar number of atoms among the plurality of samples of the data set;
computing, as the statistical characteristics, a ratio of the count of the one or more samples having the same or similar number of atoms to a total number of the plurality of samples; and
computing, based on the ratio of the count of the one or more samples to the total number of the plurality of samples, the weight corresponding to each of the plurality of samples.

16. The method of claim 13, wherein the determining of the first cutoff radius of the potential energy model comprises:
determining, for each elemental combination of atoms of the key sample, an importance value corresponding to a degree to which each elemental combination contributes to a number of edges of an atomic graph; and
determining, based on the importance value of each elemental combination of the atoms, the first cutoff radius of each elemental combination of the atoms.

17. The method of claim 16, wherein the determining of the importance value comprises:
determining an environmental descriptor of each of the atoms of the key sample;
computing an element ratio of a neighboring atom within a reference cutoff radius of each of the atoms of the key sample; and
determining, based on the environmental descriptor and the element ratio of the neighboring atom of each of the atoms, the importance value of each elemental combination of the atoms.

18. The method of claim 17, wherein the determining of the environmental descriptor of each of the atoms of the key sample comprises generating the environmental descriptor for each atom based on position coordinates and force coordinates of the respective atom and position coordinates and force coordinates of neighboring atoms within the reference cutoff radius of the respective atom, and
wherein the computing of the element ratio of the neighboring atom within the reference cutoff radius of each of the atoms of the key sample comprises:
determining a number of neighboring atoms corresponding to each element within the reference cutoff radius of each of the atoms of the key sample;
determining a total number of the neighboring atoms within the reference cutoff radius of each of the atoms; and
computing, as the element ratio of the neighboring atom corresponding to the each element, a ratio of the number of neighboring atoms corresponding to the each element to the total number of the neighboring atoms within the reference cutoff radius of each of the atoms.

19. The method of claim 13, further comprising:
generating, based on the second cutoff radius, a second atomic graph as an input to the trained potential energy model;
finetuning, using the second atomic graph, the trained potential energy model; and
obtaining a final potential energy model by repeatedly updating the second cutoff radius, generating a second atomic graph based on the updated second cutoff radius, and finetuning the trained potential energy model until an inference result of the potential energy model satisfies a defined requirement for an MD simulation.

20. A non-transitory computer-readable storage medium storing one or more programs comprising instructions that, when executed by at least one processor of an electronic device individually or collectively, cause the electronic device to perform operations comprising:
obtaining a key sample from a plurality of samples of a data set for a molecular dynamics (MD) simulation;
based on the key sample, determining a first cutoff radius of a potential energy model;
executing, using a first atomic graph generated based on the key sample and the first cutoff radius, training of the potential energy model; and
determining a second cutoff radius different from the first cutoff radius based on an inference result of the potential energy model trained using the first atomic graph, wherein the inference result of the potential energy model comprises an energy parameter and a force parameter of an atom.
